SEQUENCE LISTING (14 sequences)

SEQ ID NO: 1 - DNA, 1763 bp, Cynoscion nebulosus
  CDS (136)..(1194)
tgctgctcccgtcacggtcatctttcctccgttcaccccttcaaacaatgttatctatatcctgtacggctccagaccattcatctgccgctaatctgaagactttggattgattctaccgtctacaagtttgccatggcgacggtggtgatggagcagatcggtcgcctatttatcaacgcgcagcagctgcgtcagatccctcagcttctggagtctgccttccccacactgccttgcaccgtgaaggtgtctgatgttccctgggtgttccgcgagcgtcacatcctcactggctacagacagccggaccaaagctggcgctactactttctcaccctcttccaaaggcacaatgagactctcaatgtgtggacccatctgctggctgcattcatcatcttggtgaagtggcaggaaatctcagagacggtcgattttttgcgagaccctcatgctcagccccttttcattgtcctcctggcagccttcacctacctctccttcagcgctctcgctcatctcctctctgccaagtctgagctctcctactacaccttctacttcctcgactatgtgggggttgctgtctaccagtacggcagtgccctggcgcactactactacgccatagagaaagagtggcacactaaagtgcaagggctctttttaccggctgcagcattcttggcctggctcacttgctttggctgctgctatggcaaatatgcgagtcctgagctgcccaaggttgccaacaagctcttccaagtggtgccctcagccttggcttactgtttagacataagccctgtggttcaccgcatctacagctgctaccaggagggctgctctgatccagtcgtggcgtaccatttctaccatgtggtctttttcttaattggcgcctatttcttctgctgccctcacccagagagtttgttccctgggaagtgtgacttcatcgggcagggccaccagctcttccacgtgttcgtggtagtgtgcaccttgacgcaggtggaagcgctgcgaacagacttcacggagcgtcgccccttctacgagcgccttcacggcgatcttgcacacgatgccgttgcactcttcatcttcactgcctgctgcagcgctctcaccgctttttacgtgcgccagcgtgtacgtgcctctctacacgagaagggggagtaagatttgaaaagagtaaaaaaaaaaaaagagagagaagttgagtctaatttattttactctgtagtgaccattaattaaaaatgttttttttgtcaatgaagtttgtgacagtgacttatggttatttctcttcaatgtgatctgccaaactacagcaggctaaaacaaagggtttttgtgactatgaggaggagggagtcttataacaggtctgtagcagtataggtagtattaaacactgaaatgagtccttgtatctctactctgctctttttttttagtcggttgtttttagtcatagtctaaagaaatgtgttttacttcacgtacaaaaatacaacacttaatgtcgaggtgtgcagcaaaatgaagagactggtttcttaagatgccttttacccaattttattcagaataatttgactgttttaatcaaattgtagcttttcaaagcacccaagcattttattttttacaatttacaatcctataatactttgtgtattgcggtcagatacgtaccaattccaatccaatttaaattggtgttcttgacacacctttctgag

SEQ ID NO: 2 - protein, 352 aa, Cynoscion nebulosus
MATVVMEQIGRLFINAQQLRQIPQLLESAFPTLPCTVKVSDVPWVFRERHILTGYRQPDQSWRYYFLTLFQRHNETLNVWTHLLAAFIILVKWQEISETVDFLRDPHAQPLFIVLLAAFTYLSFSALAHLLSAKSELSYYTFYFLDYVGVAVYQYGSALAHYYYAIEKEWHTKVQGLFLPAAAFLAWLTCFGCCYGKYASPELPKVANKLFQVVPSALAYCLDISPVVHRIYSCYQEGCSDPVVAYHFYHVVFFLIGAYFFCCPHPESLFPGKCDFIGQGHQLFHVFVVVCTLTQVEALRTDFTERRPFYERLHGDLAHDAVALFIFTACCSALTAFYVRQRVRASLHEKGE

SEQ ID NO: 3 - DNA, 1262 bp, Homo sapiens
  CDS (97)..(1137)
gctgaagtttgcctgacaccatcaaccaggccctagtcacctggctttgcctttgccctgctgtgtgatcttagctccctgcccaggcccacagccatggccatggcccagaaactcagccacctcctgccgagtctgcggcaggtcatccaggagcctcagctatctctgcagccagagcctgtcttcacggtggatcgagctgaggtgccgccgctcttctggaagccgtacatctatgcgggctaccggccgctgcatcagacctggcgcttctatttccgcacgctgttccagcagcacaacgaggccgtgaatgtctggacccacctgctggcggccctggtactgctgctgcggctggccctctttgtggagaccgtggacttctggggagacccacacgccctgcccctcttcatcattgtccttgcctctttcacctacctctccttcagtgccttggctcacctcctgcaggccaagtctgagttctggcattacagcttcttcttcctggactatgtgggggtggccgtgtaccagtttggcagtgccttggcacacttctactatgctatcgagcccgcctggcatgcccaggtgcaggctgtttttctgcccatggctgcctttctcgcctggctttcctgcattggctcctgctataacaagtacatccagaaaccaggcctgctgggccgcacatgccaggaggtgccctccgtcctggcctacgcactggacattagtcctgtggtgcatcgtatcttcgtgtcctccgaccccaccacggatgatccagctcttctctaccacaagtgccaggtggtcttctttctgctggctgctgccttcttctctaccttcatgcccgagcgctggttccctggcagctgccatgtcttcgggcagggccaccaacttttccacatcttcttggtgctgtgcacgctggctcagctggaggctgtggcactggactatgaggcccgacggcccatctatgagcctctgcacacgcactggcctcacaacttttctggcctcttcctgctcacggtgggcagcagcatcctcactgcattcctcctgagccagctggtacagcgcaaacttgatcagaagaccaagtgaagggggatggcatctggtagggagggaggtatagttgggggacaggggtctgggtttggctccaggtgggaacaaggcctggtaaagttgtttgtgtctggccaaaaaaaaaaaaaaaaaaaaaa

SEQ ID NO: 4 - protein, 346 aa, Homo sapiens
MAMAQKLSHLLPSLRQVIQEPQLSLQPEPVFTVDRAEVPPLFWKPYIYAGYRPLHQTWRFYFRTLFQQHNEAVNVWTHLLAALVLLLRLALFVETVDFWGDPHALPLFIIVLASFTYLSFSALAHLLQAKSEFWHYSFFFLDYVGVAVYQFGSALAHFYYAIEPAWHAQVQAVFLPMAAFLAWLSCIGSCYNKYIQKPGLLGRTCQEVPSVLAYALDISPVVHRIFVSSDPTTDDPALLYHKCQVVFFLLAAAFFSTFMPERWFPGSCHVFGQGHQLFHIFLVLCTLAQLEAVALDYEARRPIYEPLHTHWPHNFSGLFLLTVGSSILTAFLLSQLVQRKLDQKTK

SEQ ID NO: 5 - DNA, 2221 bp, Homo sapiens
  CDS (3)..(671)
  polyA_signal (842)..(847)
  polyA_signal (1017)..(1022)
  polyA_signal (1102)..(1107)
tgcagtccaagtcagagctctcccactacaccttctactttgtggactatgttggcgtgagcgtttaccaatatggcagtgctttggctcatttcttctacagctctgaccaggcctggtatgaccggttctggcttttcttcttgccagcagctgccttctgtggctggttatcttgtgctggctgttgctatgccaaatatcgttaccggaggccttatccagtcatgaggaagatctgtcaagtggtgccagcaggtctggcttttatcctagacatcagccctgtggcacaccgtgtggcgctctgtcacctggctggctgccaggagcaagcagcctggtaccacaccctccagatcctcttcttcctggttagcgcttatttcttctcctgccccgtgcctgagaagtacttcccgggttcctgtgacatcgtgggccatgggcatcagatcttccatgcatttctgtccatctgtacgctctcccagctggaggccatcctcctggactaccaggggcggcaggagatcttcctgcagcgccatggacccctatctgtccacatggcctgcctctccttcttcttcctggctgcctgcagtgctgccaccgcagcccttctgaggcacaaagtcaaggccagactgaccaagaaagattcctgaggctggcaagtggggcaacgtgtggaggaagcccctcataatttggagaaaacttgatacaatagaagctgacttttaaggcattggcttttaaattaatacatatatccaaggatatgttacagctgcagtgtttgaaagccaaaggatttaagagttttgttgttgttaataaaaggaatactccttttccttttggatcatagcttaacaaggcacaggaagggaagggatcttgactaagattcatgagacattgaattaaggagaatcatcttcatgcctgaaaatttagcaaaattccgactatggcctccaggggcaattcctaaaagctgaatggataataaaattggactggaaagtaagtaggtggctggtcctcaccctgttggaatggctatcctactatgctgttctttggtaatggaataaattgacccaaggaccgaatttcatttggatttcaaattgtccagagtggaaaagccttcaagatgacatgatgaattactcagttcatctgatttctggtccctcctttctcgacaactataatactaacccttttctcaggataactgtctacacctggcagttttctctgaagtgctgttcactcacatccctaccttgcatggtaatataaaggactaggaagcagtcatacttccaggaaatgcttggattcatgtggacattcaggaagcttattctcatataatactaatctaaacagtactagaaattacagtgccaagagccaccaggaggcccagccaataagcatagatactatatggtatcatgggacccatctattttttaccagtggactacaggattacttgagagttatcagggctgcctaacagaccaggagatctgggggttgcaccagggaatcgccatatttgaccagcatgttttaaaagctcttggtaggattagttggttctaaggatccctctagggacctcattatttcaagaggaacccaaagtccagcctcctacatagatgctgccccacgaaggacccacaaaactaacctagttcagggttctcaggcaggcagttctgcttcagcttagagcagaacccataaaatactcaagtactgggataggcaagcatgtgtgtttactgtggattggtccctgaaggctcctttgggtgagaacatgtgaaccaggcaccctggtttgtttggagcattgctgcccagaagcttctatgggataggtggtgcttgggattgatgtgttgtggccatgcagccctccctgaggattgacttctgcactaatccagtgaaggaggctgtgtcaaaagaagggctcagaagccctcttttcagaggcaatgattcctgtcagtatgaggtcccttagttactaaaaagggacatgatttaactccagtttcatgaacctcctccgagtttactttattgtcttcaaatcttttgttttcttcctttttgtgagatttgtgggttttgtgccttataaatggaaatgtatgaacacaaaaaaaaaaaaaaaaaa

SEQ ID NO: 6 - protein, 222 aa, Homo sapiens
QSKSELSHYTFYFVDYVGVSVYQYGSALAHFFYSSDQAWYDRFWLFFLPAAAFCGWLSCAGCCYAKYRYRRPYPVMRKICQVVPAGLAFILDISPVAHRVALCHLAGCQEQAAWYHTLQILFFLVSAYFFSCPVPEKYFPGSCDIVGHGHQIFHAFLSICTLSQLEAILLDYQGRQEIFLQRHGPLSVHMACLSFFFLAACSAATAALLRHKVKARLTKKDS

SEQ ID NO: 7 - DNA, 2054 bp, Mouse
  CDS (62)..(1099)
  polyA_signal (2017)..(2022)
agacagctcctagctctgctctgaccacagttttcctgacttctcttccgggccctccggcatggcgatggcagtagcccagaagttcaaccaccttctgtccagcctgtggcacgtgggccagaagcctccgcaaccagaacctgtgtttacagtggaccgggcccaggtgccgccacttttctggaagccgtacatctatgctggctaccggccgctgcatcagaactggtgtttctacttccgcacactgtttcagcggcacaacgaggctgtgaacgtgtggacccacctcctggcggccctggctctgctgctgcggctgatcggcttggcggcaagtgtggacttccgggaagaccctcacgcgctgcccctcttcttcatcgtcttggcctccttcacctacctctcgttcagtgctgtggctcacctcctgcaggccaagtcggagttctggcattacagcttcttcttcttggactatgtgggtgtggccgtgtaccagtttggcagtgccctggcacacttctactatgccatagagccgtcctggcatgacaaggtgcaggctattttcctgcccacggccgccttcctggcctggctttcctgcgctggctcctgctacaacaagtacagccagaagccgggtctgctggggcgcattttccaggaggcgccatcggcgctagcctatgtgctggacatcagtcccgtgttgcaccgcatcatagtgtctcccctccctgccgaggaggatcccgctcttctctatcacaaatgccaagtggttttcttccttctagctgctgcctttttctccacggttatgcctgagagttggttccccggcagctgtcacatctttgggcagggacaccaagttttccatgtccttttggtgctgtgcactctggcccagctagaggcggtgacactggattatcaggcccggaggggcatatacgagcctctgcacgcccgttggccccgcaacttctctggcctcttcctgctcaccgtggccagcagcagcctcactgcgctcctcctcagccagctggtgcggcgcaaactccatcagaagaccaagtgaaagcgggtgggtgggagctggcagggtgagagaaggtgctagggacaaaagcctggacttggctccagatcggaacaaggcctggtaaagctggttctatctggcaggccatgactccctgcatacaagctcaatggccaaagtgatgctgctagccaatccttgggcctgcagattggctgggagctgtagagccttgctcagttccctgggaggggcagagatgaaggactcctgactgcccctcccttgccgcctatcagggctaagggttggagttcagctttggtcaccacactggttaggggttttcaggttgctgggcgagaagactcaggccagtgtcaaatttgagcagagagggagtggaagtcttagcagccaccacctaccagagctcactggtggagggaaaagaaacaggcccaacgatgggtagtgttttaacttcactgtcccctgcaccacagcctggatcgctgggttccagagagtctccaaagataagagatcccctctcctgcccacatccctgcatcacagcccagccagaaagcctcacagctagcattggcctgtatctcttgcacactgtttccagcttctccccacaactcatttatgattttgagatatgactccttagctcactgtttcctggggctccaggctttagaaccgttaggaattgtcaaggaaaactcaaagtgctgactagtcagcactgcagggcacgcagagccttggcttaggaagggtgaggagtttgcaggctgagcttagaagggcgttgcaggcaaaagggaagcctgtaccgagattcgagatgcagagatgcagggagttcaggggggctaaaggcaagtttggtccctggggctgagagccaaggctggtccctgatatgccctagattcaataaaatgactatgatattgaaaaaaaaaaaaaaaaa

SEQ ID NO: 8 - protein, 345 aa, Mouse
MAMAVAQKFNHLLSSLWHVGQKPPQPEPVFTVDRAQVPPLFWKPYIYAGYRPLHQNWCFYFRTLFQRHNEAVNVWTHLLAALALLLRLIGLAASVDFREDPHALPLFFIVLASFTYLSFSAVAHLLQAKSEFWHYSFFFLDYVGVAVYQFGSALAHFYYAIEPSWHDKVQAIFLPTAAFLAWLSCAGSCYNKYSQKPGLLGRIFQEAPSALAYVLDISPVLHRIIVSPLPAEEDPALLYHKCQVVFFLLAAAFFSTVMPESWFPGSCHIFGQGHQVFHVLLVLCTLAQLEAVTLDYQARRGIYEPLHARWPRNFSGLFLLTVASSSLTALLLSQLVRRKLHQKTK

SEQ ID NO: 9 - DNA, 1496 bp, Mouse
  CDS (347)..(1411)
  polyA_signal (1476)..(1481)
ctggcgcaggatgcaggtaaatgttgagacatcccataatgattgtatttacctaagaagaaaaatagatttcctgaaatcccagcacttgctgatatcaaagctgcattaattctacgcacaggagttcctcaggtcctgattgaagcacttgaagtagcttatctgccagctctacctccctgcttgtttgcagaaaccccgtggttcatctcacacctcagctggagcgtggattgcacatctgtcataatcccactacacgtctctctatggaacactacagacgctaatctctgcaaaggttgtcttactgagccccgaggagcccagggccaccgctgtcatgacgactgccatcctagagcgcctgagcaccctgtcaatgagcgggcagcagctgcgccgtctgcccaagattctggaagaaggacttcccaagatgccatgcaccgtcccagaaaccgacgtgccccagctctttagggagccgtacatccacgcgggctaccgccccacggggcacgagtggcgttactacttcttcagcctctttcagaagcacaacgaggtggtcaacgtctggacccacttgctggctgctctagcggtccttttgcgattctgggcctttgtggaggcaggggcattgcagtgggcctctccccacaccctccccctgctcctctttgtcctgtcgtccatcacttacctcacctgcagcctcttggcccacctgctgcagtccaaatcagagctgtcccactacacgttctactttgtggactacgtcggggtcagcgtctaccagtatggcagcgccctggctcacttcttctatagctccgaccaggcctggtatgagctattttggctttttttcctgccggcggctgctttctgtggctggctctcctgcgctggctgttgctatgccaagtatcgctaccgacggccttatccagttatgaggaagatctgtcaagtagtaccagcagggctggccttcgtcctagacatcagcccggtggcacaccgcgtggctctctgccacctggccggttgccaggagcaggcggcctggtaccacaccctccagatcctcttcttcctcgtcagcgcatacttcttctcgtgccctgtccccgagaagtacttccccggttcctgtgacattgtgggccacggacatcaaatcttccacgccttcctgtccgtctgcacgctctcccagctggaggccattcttctggactaccagggacgccatgagatcttcctccagcgccacggtcccctgtctgtgtacagcgcctgcctctcctttttcgttttagctgcctgcagtgcggccaccgcctccctcctgaggcacaaggtcaaggacagactgattaagaaagattcctgaggtccttcaaatgaggaaaggtgtggaggtgggctactgtgacttggagaaaacttgacccaataataaaaaaaaaaaaaaaaaa

SEQ ID NO: 10 - protein, 354 aa, Mouse
MTTAILERLSTLSMSGQQLRRLPKILEEGLPKMPCTVPETDVPQLFREPYIHAGYRPTGHEWRYYFFSLFQKHNEVVNVWTHLLAALAVLLRFWAFVEAGALQWASPHTLPLLLFVLSSITYLTCSLLAHLLQSKSELSHYTFYFVDYVGVSVYQYGSALAHFFYSSDQAWYELFWLFFLPAAAFCGWLSCAGCCYAKYRYRRPYPVMRKICQVVPAGLAFVLDISPVAHRVALCHLAGCQEQAAWYHTLQILFFLVSAYFFSCPVPEKYFPGSCDIVGHGHQIFHAFLSVCTLSQLEAILLDYQGRHEIFLQRHGPLSVYSACLSFFVLAACSAATASLLRHKVKDRLIKKDS

SEQ ID NO: 11 - DNA, 2254 bp, Pig
  CDS (156)..(1208)
  polyA_signal (2204)..(2209)
agccgggcacatcctctcgaagctccgttcggccgcaggggacagaaaccagtcaagtttgcctgacatccatcagccagggcctggacacctggtctcagcccagctctgcctgccttgctgtgtgatctaggctccctgccccacccacagccatggccacgatggtggcccagaagctcagccacctcctgcccagtttgcgacaggtccatccggagcctcagccgtctgtgcacccagagcctgtgttcactgtggaccgagctgaggtgccgcccctcttctggaagccatacatctacgtgggctaccggccgctgcatcagacctggcggttctacttccgcacactgttccagcagcacaacgaggcggtgaacgtctggacccacctgctggctgccctggtgctgctgctgcggctggccatctttgtggggaccgtggacttctggggagacccacatgccctgcccctcttcatcattgtcctggcctccttcacctacctctccctcagtgccttggctcacctcctgcaggccaagtctgagttctggcattacagcttcttcttcctggactatgtgggtgtggccgtgtaccagtttggcagtgccctggcgcacttctactatgccatcgagcccgcctggcatgcccaggtgcagaccattttcctgcccatggctgcctttctcgcctggctgtcctgcactggctcctgctacaacaagtacatccagaaacccggcctgctgggccgcacttgccaggaggtgccctcagcgctggcctacgcgctggacatcagccccgtggcgcaccgcatcctcgcgtccccggaacctgccacagacgacccggctcttctctaccacaaatgccaggtggtcttctttctactggctgctgctttcttctctgccttcatgcctgagcgctggttccctggcagctgtcacatctttgggcagggccaccagctcttccatgttttcttggtgctgtgcacgctggctcagctggaggctgtggcgctagactatgaggcccggcggcccatctatgagcctctgcatacccgctggccccacaacttctccggcctcttcttgctcaccgtaggcagcagcatccttaccgcgttcctcctgagccagctggtacgacgcaaactcgatctcgatcggaagacccagtgaacggtggggtggcagctaggagggagggaggtgtaatgggggcccaagggtccgggcttggctccagatgggaacaagccctggtaaagttgtttgtgtctggctcgcaatgactttctgtgtatgccccagctgccaagggtggcactggccagtctttgaatttgcggattggctggagatgttggggtccagtcctgggcctgtcccagctccctgccctgcgagagggaaagaaggatttgggacacccaggtttgcctccctgcattgtcttttacttagaagtgaggatgggggatcagctggggccaagaccctggtccagggcttccagacaatccgggagtgggtgaagttgggatttaggtcagagtcagatgtgagctgagagatgggaagcgtcagcaataccccctctattcagatttctctggtggatagggaaagggcaggcccagaatgtgtgcagaaccttgaaccccagcccggaattttagtgttccagagcgtccccaaaagtagaagataatgccaggtagaaatggatcccatccagtgtcccatacttttttagtccctgtcccagacagcgagccccaaccctcctagctcaaacctctgtgtcctacacatcctgttcccagcaactttcccagttcccttactcacgattcagtgtttatccattcattgtttcttgggccttttctcagagccaggccactgactgggccctgtggatcaatacaggatggtgaaggctttaagatcggaatgaactgtaaggggaagcactgaggagggaataagtagtactgcctgggaccctcagggtcttgggttgggaagggtgactaggagtttgcagatggaaaaggagaagggtattccaggaagagggaaaaacctgtccgaagactaagaggtgtggaagggagagttcactggggttggagtgaggggtagtttgggccttgacatgtcctgggtgcaataaagtgactgtggtaaaaaaaaaaaaaaaaaaaaaaaaaaaaaaaaaa

SEQ ID NO: 12 - protein, 350 aa, Pig
MATMVAQKLSHLLPSLRQVHPEPQPSVHPEPVFTVDRAEVPPLFWKPYIYVGYRPLHQTWRFYFRTLFQQHNEAVNVWTHLLAALVLLLRLAIFVGTVDFWGDPHALPLFIIVLASFTYLSLSALAHLLQAKSEFWHYSFFFLDYVGVAVYQFGSALAHFYYAIEPAWHAQVQTIFLPMAAFLAWLSCTGSCYNKYIQKPGLLGRTCQEVPSALAYALDISPVAHRILASPEPATDDPALLYHKCQVVFFLLAAAFFSAFMPERWFPGSCHIFGQGHQLFHVFLVLCTLAQLEAVALDYEARRPIYEPLHTRWPHNFSGLFLLTVGSSILTAFLLSQLVRRKLDLDRKTQ

SEQ ID NO: 13 - DNA, 2722 bp, Pig
  CDS (73)..(1137)
gcctcctgcgggaagccgggatcctgggctgggagtgcgggctggctctgcgggttgtatccctgtgcagccatgacaaccgccatcctgcagcgcctaagcaccctgtcggtgagcgggcagcatctgcgccgcctgcccaagatcctggaggacgggctgcccaagatgcctggcactgtgcccgagaccgacgtgccccagctcttccgggagccttacatccgcgccgggtaccgccccatcggccacgagtggcgttactacttcttcagcctctttcagaaacacaacgaggtggtcaacgtgtggacccacctgctggcggccctggccgtcctcctgcggttctgggccttcgtggagaccgagggcctgccctggacctctgctcacaccctgcccctgctcctctacgtcctgtcctccatcacttacctcaccttcagcctgctggcccacctgctgcagtccaagtccgagctctcccactacaccttctacttcgtggactacgtgggcgtgagcgtgtaccagtacggcagcgccctggtccacttcttctacgcctccgaccaggcctggtacgagcgcttctggctcttcttcctgcccgcggccgccttctgcggctggttatcttgcaccggctgctgctacgccaagtaccgttaccgccggccctacccggtcatgaggaaggtctgccaagtggtgcccgcggggctggccttcatcctggacatcagccccgtggcgcaccgcgtggccctgtgccacctgtctggctgccaggagcaggccgcgtggtaccacaccctccagatcgtcttctttctggtcagcgcctacttcttctcctgcccagttccggagaagtactttcccggttcctgtgacatcgtgggccacggccatcagatcttccacgcctttctgtccatctgcacactctctcagctggaggccatcctcttggactacaaggggcgacaggagatcttcctgcaccgtcacagtcccctgtccatctacgccgcctgcctctccttcttcttcctggtggcctgcagcggggccactgcagccctcctgcgggaaaaaatcaaggccagactgagcaagaaggattcctgaggccagagggtggggcgaggtgtggaggcaaataggagttgacttttcgtttttaaaagcaggcttttaaataggtacatatttccaaggatggctatggctcgcgaagagttttattgttatggttgttaatgaaaggagcattccttttccttcggggccatagccttcccagacaggaagggaagggaccttggcaaagattcagcagacactaaattaagaaccatctacatgcctgaaaattcagtggacttctgactgcaacctccaggagcaaaccccagaccaaaggggtcattcacttggactggaaagtgaggtggctggaccacccttgaaatgggcattcaactgtgatgcggctcttgagtgattagctacattgactcaaggacccgaattcatttggatttcagattttcagattggtggagaagtcggactcttcaggaagattcagtgaaacccatagacgtaatgaatgactccattcccctgacttttgtccccttctctgacccactccagcactgacccttttctcaggatgatgtctgctgggcagttgtctccgcagtgctgttcgctcccacctgtaccttgtatggtgatgtcaaggacagcggagcagccatccttccaggaatcactcggatccacacgaaccttcaggaagtttacttcgcatctgatactaatgtacgtggtactggagactgcagtgccaagagccaccggggggccccgccagcaagcatggataccatttggagccatgggacccttccattttctacctgtgcattgcaggattactcgagatctctcttagggctgccttacctgctagaagatccagagctttctccagaggatcttctccagattttgaccagcatgttcaaaagtgaattggttctaagattcccactagggatctgctgattttaagaggaacccgaggtccagcctcttaaatagatgctgccccatgaaggactcacagaactagcctagttcagggtccttagggagacagttgtacctcagcttagagcagcttaacttcagcatgagtgtgtgctcatggtgggttggtccccgaaggctccattggctgaggatggtggaccaggggctcctgcaggcagaatgttctgtttaatgatgtggctgcccagcagcttccatgggatagaaggggacctgcggctgatgtgttctggccacaaagctttccctgaggactgccttccgcgctgatccagtgaagggggctctggtgacaccaggtctgtgggaaccccaccaccatcaccatgggctgcatttcctctcagtataaggtttctgtttactgaaaaggaaagtggtttccactccagtttgatgaacctcctctgttactacattactctgttgtctttacatctttgttttcttcctttctgttgagagatgtgtggggtttatgccctataaatggaaacatatgaacacttaatccatggccatgtggaatttttggggttattgggataaaaaaaaaaaaaaaaaaaaaaaaaaaaaaaaaaaaaaaaaa

SEQ ID NO: 14 - protein, 354 aa, Pig
MTTAILQRLSTLSVSGQHLRRLPKILEDGLPKMPGTVPETDVPQLFREPYIRAGYRPIGHEWRYYFFSLFQKHNEVVNVWTHLLAALAVLLRFWAFVETEGLPWTSAHTLPLLLYVLSSITYLTFSLLAHLLQSKSELSHYTFYFVDYVGVSVYQYGSALVHFFYASDQAWYERFWLFFLPAAAFCGWLSCTGCCYAKYRYRRPYPVMRKVCQVVPAGLAFILDISPVAHRVALCHLSGCQEQAAWYHTLQIVFFLVSAYFFSCPVPEKYFPGSCDIVGHGHQIFHAFLSICTLSQLEAILLDYKGRQEIFLHRHSPLSIYAACLSFFFLVACSGATAALLREKIKARLSKKDS

(19) United States
(12) Patent Application Publication
(10) Pub. No.: US 2003/0170791 A1
(43) Pub. Date: Sep. 11, 2003

(54) Gene family encoding membrane steroid receptors

(76) Inventors: Yong Zhu, Greenville, NC (US); Peter Thomas, Portland, TX (US)

Correspondence Address: Sanford E. Warren, Jr., GARDERE WYNNE SEWELL LLP, Suite 3000, 1601 Elm Street, Dallas, TX, 75201, US

(21) Appl. No.: 10/167,237

(22) Filed: Jun. 11, 2002

Related U.S. Application Data
(60) Provisional application No. 60/297,726, filed on Jun. 12, 2001.

(51) Int. Cl. A61K 38/17; C12P 21/02; C12N 5/06; C07K 14/72; C07H 21/04

(52) U.S. Cl. 435/69.1; 435/320.1; 435/325; 530/350; 536/23.5

(57) ABSTRACT

A family of genes that encode membrane steroid receptors is identified in mammals and fish. A putative progestin membrane receptor (mPR) from a fish ovary was cloned, expressed and characterized. Database searching with the fish ovary mPR lead to the discovery of a new family of steroid membrane receptor genes with highly conserved sequences and structures similar to fish mPR in human, mouse and swine tissues. The cDNA sequence for each of these mammalian genes was determined. The cDNAs appear to be members of a novel gene family, not closely related to any previously characterized vertebrate genes.

This application claims priority from U.S. Provisional Application No. 60/297,726, filed on Jan. 12, 2001.

### FIELD OF THE INVENTION

The present invention relates to membrane steroid receptors and, in particular, to a family of genes encoding membrane steroid receptors.

### BACKGROUND OF THE INVENTION

The research carried out in the subject application was supported in part by grants from the National Science Foundation (NSF Number IBN-9980353) titled CLONING SEQUENCING AND EXPRESSION OF A STEROID MEMBRANE RECEPTOR.

Over the past 20 years, convincing evidence has been obtained that steroids exert non-genomic and rapid actions ranging from seconds to minutes by binding to specific steroid receptors located on the plasma membrane. The cell-surface mediated actions of steroids include rapid changes in electrolyte movement across cellular membranes and rapid second messenger signaling and MAP K activation.

Specific membrane receptors for estrogens have been identified in several tissues including the mammalian brain, pituitary, liver, uterus, testis, heart and vascular system, for androgens in the testis and vascular tissues, for corticosteroids in the kidney, colon, liver, brain and lymphocytes, and for progestins in the brain, sperm and oocytes. No information is available, however, on the structure of any membrane steroid receptors (mSR).

### SUMMARY OF THE INVENTION

The present inventors recognized that the isolation and expression of genes for membrane steroid receptors make possible an understanding of the molecular basis for the rapid, membrane-mediated actions of steroids. Such understanding will have a major impact in many diverse areas of biology and medicine including reproduction, endocrinology, neuroendocrinology, behavior, mental health, child development as well as cancer, diseases and disorders of endocrine, cardiovascular and central nervous systems.

The present invention includes the isolation, description and characterization of a trans-species family of genes encoding membrane steroid receptors. The genes of the present invention have no similarity with any other known genes in available databases. Genes for steroid membrane receptors have not been characterized previously in any vertebrate species.

Using an approach developed by the present inventors, a putative progestin membrane receptor (mPR) from a fish ovary was cloned, expressed and characterized. Database searching with the fish ovary mPR lead to the discovery of a new family of steroid membrane receptor genes with highly conserved sequences and structures similar to fish mPR in human, mouse and swine tissues. The cDNA sequence for each of these mammalian genes was determined. The cDNAs appear to be members of a novel gene family, not closely related to any previously characterized vertebrate genes.

The deduced amino acid sequences (SEQ ID NOS: 2, 4, 6, 8, 10, 12, 14, 16) of the proteins encoded by the mPR genes of the present invention show seven (7) transmembrane domains by hydrophilicity and transmembrane analysis, which is characteristic of G protein-coupled membrane receptors. Northern blot analyses showed that the transcript of fish PR was expressed in the gonads, brain and pituitary but could not be detected in liver, heart, spleen, gills, and scales. Expression of human mSR genes was obtained in tissues of brain, gonads, placenta, kidney, intestine and pituitary. The result of steroid binding studies, including analyses of Scatchard, saturation, steroid specificity, and association and dissociation kinetics, with the recombinant fish PR protein indicates that the gene encodes a membrane progestin receptor. The steroid binding properties of several of mammalian mSR have been confirmed using the recombinant expression system.

These closely related vertebrate genes encode for a family of receptors located on the surface of target cells that bind and mediate the rapid, non-genomic actions of steroid hormones. The steroid membrane receptor genes are likely to have applications in medicine and agriculture.

In particular, the present invention includes an isolated nucleic acid sequence, and complements and homologues thereof. The isolated sequence hybridizes under stringent conditions to a hybridization probe. The hybridization probe provides a nucleotide sequence selected from the group consisting of: SEQ ID NO: 1, SEQ ID NO: 3, SEQ ID NO: 5, SEQ ID NO: 7, SEQ ID NO: 9, SEQ ID NO: 11, SEQ ID NO: 13 and SEQ ID NO: 15.

Further, the present invention includes a method for identifying membrane steroid receptors and generating antibodies to the receptors. The method includes the steps of forming a complex between solubilized membrane proteins and an antibody and an antigen that specifically binds to the antibody. A steroid exposed to the complex specifically binds to the complex. A receptor to which the steroid is specifically bound may is then identified. The method may also include generating antibodies to the receptor.

In summary, the present invention includes a family of genes that encode membrane steroid receptors in mammals and fish.

### BRIEF DESCRIPTION OF THE DRAWINGS

For a more complete understanding of the features and advantages of the present invention, reference is now made to the detailed description of the invention along with the accompanying figures in which corresponding numerals in different figures refer to corresponding parts and in which:

FIG. 1 is a bar graph illustrating the solubilization of seatrout ovarian membrane progestin (20β-S) receptor with triton-X 100 at a concentration of 12 mM;

FIG. 2A is a plot graph of a saturation analysis of progestin receptor.

FIG. 2B is a Scatchard plot graph of specific binding of [³H] 20β-S binding to solubilized progestin receptor.

FIG. 3 is a plot graph illustrating the time course of association and dissociation of [³H] 20β-S binding to solubilized progestin receptor.

FIG. 4 is a chromatographic graph illustrating the elution profile of solubilized progestin (20β-S) receptor on DEAE column showing peak of progestin (20β-S) binding activity. The insert shows silver-stained SDS-PAGE of pooled fractions.

FIG. 5 is western blots of three (3) monoclonal antibodies (PR10-1, PR53-4, PR64-4) binding to solubilized seatrout ovarian membrane protein.

FIG. 6 is a plot graph of the hydrophobicity profile for the amino acid sequence of a membrane steroid receptor (SEQ ID NO: 2). Seven transmembrane domains are identified. The cDNA clone was obtained from a seatrout ovarian cDNA library screened with PR10-1 antibody.

FIG. 7 is a cross-sectional depiction of a model for the insertion of seatrout mPR. Each circle represents one amino acid residue. Filled circles indicate conserved identical residues between fish mPR and human protein (AK000197). Sites of potential N-linked glycosylation are noted.

FIG. 8A is a bar graph depicting the steroid binding specificity of the seatrout recombinant mPR produced in E. coli.

FIG. 8B is a bar graph of the control protein of FIG. 8A.

FIG. 9 is a plot graph of competition curves of steroid binding to various steroids to seatrout mPR produced in E. coli.

FIG. 10 is a plot graph of competition curves of binding of various C21 steroids to seatrout mPR produced in E. coli.

FIG. 11 is a representative plot graph of specific [³H]progesterone binding to the seatrout mPR produced in E. coli (0). [³H]progesterone binding to control protein in E. coli with a vector (+). The insert is a Scatchard analysis of the specific binding.

FIG. 12 is a plot graph of the time course of association and dissociation of [³H]P₄ binding to the seatrout mPR.

FIG. 13 is Northern blot analysis of the seatrout mPR receptor RNA is seatrout ovary and brain.

FIG. 14 is a schematic comparison of deduced amino acid sequence homology of seatrout mPR and three (3) novel human proteins within extracellular (opaque segments), transmembrane (shaded segments), and cystolic domains (open segments). The number above (human) and below (seatrout) the boxes indicate the number of amino acids from the N-terminal. The number inside the boxes indicates the percent identity of amino acids of mPR of human compared to seatrout.

FIG. 15 is amino acid sequences of mPR in various tissues from human testis (SEQ ID NO; 4), human brain (SEQ ID NO: 6), mouse brain (SEQ ID NO: 8), mouse testis (SEQ ID NO: 10), pig embryo (SEQ ID NO: 12), pig intestine (SEQ ID NO: 14) and seatrout (SEQ ID NO: 2) and shows the alignment of the respective sequences.

FIG. 16 is amino acid sequences of human testicular mSR (SEQ ID NO: 4), human brain mSR (SEQ ID NO: 6) and human kidney (SEQ ID NO: 16) aligned with seatrout mSR (SEQ ID NO: 2).

FIG. 17 is a phylogram illustrating the results of phylogenetic analysis of membrane steroid receptor (mSR) cDNA-coding sequences.

FIG. 18 is a diagram of an exemplary multiple human tissue array.

FIG. 19 is a photograph of dot hybridization of human mSR mRNA with multiple human tissue arrays. Panel I: hybridized with a human kidney mSR (SEQ ID NO: 16) probe containing late-part of the coding region; Panel II: hybridized with a human kidney mSR (SEQ ID NO: 16) probe containing 3-UTR; Panel III: hybridized with human brain mSR (SEQ ID NO: 6) probe; Panel IV: hybridized with a human testis mSR (SEQ ID NO: 4) probe.

FIG. 20 is a photograph of Northern hybridizations of human mSR probes to human multiple tissue Northern blots. Panel A: hybridized to a human kidney (SEQ ID NO: 16) mSR probe; Panel B and D: hybridized to a human testes mSR (SEQ ID NO: 4) probe; Panel C: hybridized to a human brain mSR (SEQ ID NO: 6) probe.

FIG. 21 is a plot graph depicting saturation analysis of specific [³H]P₄ binding to recombinant mouse testicular mSR rmSR). Inset: Scatchard plot of same.

FIG. 22 is a plot graph depicting saturation analysis of specific [³H]P₄ binding to recombinant human testicular mSR. Inset: Scatchard plot of same.

FIG. 23 is a plot graph depicting saturation analysis of specific [³H]P₄ binding to recombinant human kidney mSR. Inset: Scatchard plot of same.

FIG. 24 is a plot graph of the time course of association and dissociation of [³H]P₄ binding to the human kidney rmSR.

FIG. 25 is a plot graph of competition curves of steroid binding to various progestins to human kidney membrane steroid receptor produced in E. coli.

FIG. 26 is a plot graph of competition curves of steroid binding to various steroids to human mSR produced in E. coli.

FIG. 27 is a plot graph of competition curves of steroid binding of antihormones to the human kidney mSR produced in E. coli.

FIG. 28 is a table comparing the percent of sequence identity between steroid membrane receptors of the present invention in human, mouse, pig and seatrout. Upper right corner: comparison of cDNA sequences. Lower left corner: comparison of deduced amino acid sequences.

### DETAILED DESCRIPTION OF THE INVENTION DEFINITIONS

While the making and using of the various embodiments of the present invention are discussed in detail below, it should be appreciated that the present invention provides many applicable inventive concepts that can be embodied in a wide variety of specific contexts. The specific embodiments described herein are merely illustrative of specific ways to make and use the invention and do not delimit the scope of the invention nor the scope of the claims appended hereto.

Definitions

To facilitate the understanding of this invention, a number of terms are defined below. Terms defined herein have meanings as commonly understood by a person of ordinary skill in the areas relevant to the present invention. The definition of some terms may be provided or further explicated in the specification where such term may be used in the description of the invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

As used herein the terms “protein,” “peptide” and “polypeptide” refer to compounds comprising amino acids joined via peptide bonds and are used interchangeably.

The terms “gene sequences” or “native gene sequences” are used to indicate DNA sequences encoding a particular gene which contain the same DNA sequences as found in the gene as isolated from nature. In contrast, “synthetic gene sequences” are DNA sequences which are used to replace the naturally occurring DNA sequences when the naturally occurring sequences cause expression problems in a given host cell. For example, naturally occurring DNA sequences with codons that are rarely used in a host cell may be replaced (e.g., by site-directed mutagenesis) such that the synthetic DNA sequence represents a more frequently used codon. The native DNA sequence and the synthetic DNA sequence will preferably encode the same amino acid sequence.

As used herein, the term “gene” means the deoxyribonucleotide sequences comprising the coding region of a structural gene and the including sequences located adjacent to the coding region on both the 5′ and 3′, ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the MRNA are referred to as 3′ non-translated sequences, these sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences that direct the termination of transcriptions, post-transcriptional cleavage and polyadenylation.

As used herein, the term “structural gene” refers to a DNA sequence coding for RNA or a protein. In contrast, “regulatory genes” are genes that encode products that control the expression of other genes (e.g., transcription factors).

As used herein the term “coding region” when used in reference to structural gene refers to the nucleotide sequences that encode the amino acids found in the nascent polypeptide as a result of translation of an mRNA molecule. The coding region is bounded, in eukaryotes, on the 5′ side by the nucleotide triplet “ATG” which encodes the initiator methionine and on the 3′ side by one of the three triplets which specify stop codons (i.e., TAA, TAG, TGA).

The term “portion,” as used herein, with regard to a protein nucleic acid (as in “a portion of a given protein”) refers to fragments of that protein or nucleic acid. The fragments may range in size from four amino acid residues to the entire amino acid sequence minus one amino acid or nucleic acids ranging from 10 nucleotides to the entire genomic sequence.

“Nucleic acid sequence” or “nucleotide sequence” as used herein refers to an oligonucleotide or polynucleotide, and fragments or portions thereof, and to DNA or RNA of genomic or synthetic origin that may be single- or double-stranded, and represent the sense or antisense strand.

Similarly, “amino acid sequence” as used herein refers to an oligopeptide, peptide, polypeptide, or protein sequence, and fragments or portions thereof, and to naturally occurring or synthetic molecules. Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

A “composition comprising a given nucleotide sequence” as used herein refers broadly to any composition containing the given nucleotide sequence. The composition may comprise an aqueous solution. Compositions comprising nucleotide sequences encoding proteins or fragments thereof, may be employed as hybridization probes. Nucleotide sequences are typically employed in an aqueous solution containing salts (e.g., NaCl), detergents (e.g., SDS) and other components (e.g., Denhardt's solution, dry milk, salmon sperm DNA, etc.).

A “deletion,” as used herein, refers to a change in either amino acid or nucleotide sequence in which one or more amino acid or nucleotide residues, respectively, are absent.

An “insertion” or “addition” as used herein, refers to a change in an amino acid or nucleotide sequence resulting in the addition of one or more amino acid or nucleotide residues, respectively, as compared to the naturally occurring molecule.

A “substitution,” as used herein, refers to the replacement of one or more amino acids or nucleotides by different amino acids or nucleotides, respectively.

The term “biologically active,” as used herein, refers to a protein having structural, regulatory or biochemical functions of a naturally occurring molecule.

Likewise, “immunologically active” refers to the capability of the natural, recombinant synthetic proteins, or any oligopeptide thereof, to induce a specific immune response in appropriate animals or cells and to bind with specific antibodies.

As used herein, the term “purified” or “to purify” refers to the removal of contaminants from a sample. For example, proteins of interest are purified by removal of contaminating proteins; they are also purified by the removal of substantially all proteins that are not of interest. The removal of non-immunoglobulin proteins and/or the removal of immunoglobulins that do not bind protein results in an increase in the percent of protein of interest-reactive immunoglobulins in the sample. In another example, recombinant polypeptides are expressed in bacterial host cells and the polypeptides are purified by the removal of host cell proteins; the percent of recombinant polypeptides is thereby increased in the sample.

The term “substantially purified,” as used herein, refers to nucleic or amino acid sequences that are removed from their natural environment, isolated or separated, and are at least 60% free, 75% free, or even 90% free from other components with which they are naturally associated.

The term “recombinant DNA molecule” as used herein refers to a DNA molecule that includes segments of DNA joined together using molecular biological techniques.

The term “recombinant protein” or “recombinant polypeptide” as used herein refers to a protein molecule that is expressed from a recombinant DNA molecule. The term “native protein” as used herein refers to a protein isolated from a natural source as opposed to the production of a protein by recombinant means.

The term “modulate,” as used herein, refers to a change or an alteration in, e.g., the measured biological activity of a protein (e.g., mammalian proteins). Modulation may be an increase or a decrease in protein, protein activity, a change in binding characteristics, or any other change in the biological, functional, or immunological properties of a protein.

The term “antagonist” refers to molecules or compounds that inhibit the action of a composition (e.g., a protein). Antagonists may or may not be homologous to the targets of these compositions in respect to conformation, charge or other characteristics. In one embodiment, antagonists prevent the functioning of proteins. It is contemplated that antagonists may prevent binding of a protein and its target(s). However, it is not intended that the term be limited to a particular site or function.

The term “derivative,” as used herein, refers to the chemical modification of a nucleic acid encoding a protein (in particular, mammalian proteins), or the encoded protein. Illustrative of such modifications would be replacement of hydrogen by an alkyl, acyl or amino group. A nucleic acid derivative encodes a polypeptide that retains at least a portion of the biological characteristics of the natural molecule.

A “variant” of a protein, as used herein, refers to an amino acid sequence that is altered by one or more amino acids. The variant may have “conservative” changes, wherein a substituted amino acid has similar structural or chemical properties (e.g., replacement of leucine with isoleucine). More rarely, a variant may have “nonconservative” changes (e.g.. replacement of a glycine with a tryptophan). Similar minor variations may also include amino acid deletions or insertions, or both. Guidance in determining which amino acid residues may be substituted, inserted, or deleted without abolishing biological or immunological activity relay be found using computer programs well known in the art, for example, DNASTAR software or conserved sequence comparisons between alleles or even across species.

“Alterations” in a polynucleotide, as used herein, comprise any alteration in the sequence of polynucleotides, including deletions, insertions and point mutations that may be detected using hybridization assays. Included within this definition is the detection of alterations to the genomic DNA sequence that encodes a protein (e.g., by alterations in the pattern of restriction fragment length polymorphisms) capable of hybridizing to a particular sequence, the inability of a selected fragment to hybridize to a sample of genomic DNA (e.g., using allele-specific oligonucleotide probes), and improper or unexpected hybridization, such as hybridization to a locus other than the normal chromosomal locus for the polynucleotide sequence encoding a protein (e.g., using fluorescent in situ hybridization [FISH] to metaphase chromosomes spreads).

A “consensus gene sequence” refers to a gene sequence that is derived by comparison of two or more gene sequences and which describes the nucleotides most often present in a given segment of the genes; the consensus sequence is often referred to as the “canonical” sequence. In some embodiments, “consensus,” refers to a nucleic acid sequence that has been resequenced to resolve uncalled bases, or which has been extended using any suitable method known in the art, in the 5′ and/or the 3′ direction and resequenced, or which has been assembled from the overlapping sequences of more than one clone using any suitable method known in the art, or which has been both extended and assembled.

The term “sample,” as used herein, is used in its broadest sense. The term encompasses biological sample(s) suspected of containing nucleic acid encoding a protein or fragments thereof and may include a cell, chromosomes isolated from a cell (e.g., a spread of metaphase chromosomes), genomic DNA (in solution or bound to a solid support such as for Southern analysis), RNA (in solution or bound to a solid support such as for northern analysis), cDNA (in solution or bound to a solid support), an extract from cells or a tissue, and the like.

As used herein, the term “overproducing” is used in reference to the production of polypeptides in a host cell, and indicates that the host cell is producing more of the polypeptide by virtue of the introduction of nucleic acid sequences encoding the polypeptide than would be expressed by the host cell absent the introduction of these nucleic acid sequences. To allow ease of purification of polypeptides produced in a host cell it is preferred that the host cell express or overproduce the polypeptide at a level greater than 1 mg/liter of host cell culture.

As used herein, the term “fusion protein” refers to a chimeric protein containing the protein of interest joined to an exogenous protein fragment. The fusion partner may enhance solubility of the protein as expressed in a host cell, may provide an “affinity tag” to allow purification of the recombinant fusion protein from the host cell or culture supernatant, or both. If desired, the fusion protein may be removed from the protein of interest prior to immunization by a variety of enzymatic or chemical means known to the art.

As used herein, the term “affinity tag” refers to any structure or compound which facilitates the purification of a recombinant or fusion protein from a host cell, host cell culture supernatant, or both. As used herein, the term “flag tag” refers to short polypeptide marker sequence useful for recombinant protein identification and purification.

As used herein, the term “chimeric protein” refers to two or more coding sequences obtained from different genes that have been cloned together and that, after translation, act as a single polypeptide sequence. Chimeric proteins are also referred to as “hybrid proteins.” As used herein, the term “chimeric protein” refers to coding sequences that are obtained from different species of organisms, as well as coding sequences that are obtained from the same species of organisms. As used herein, the term “protein of interest” refers to the protein whose expression is desired within an expression vector or a host cell.

As used herein “soluble” when in reference to a protein produced by recombinant DNA technology in a host cell, is a protein which exists in solution in the cytoplasm of the host cell, if the protein contains a signal sequence. the soluble protein is secreted into the culture medium of eukaryotic cells capable of secretion or by bacterial hosts possessing the appropriate genes. In contrast, an insoluble protein is one which exists in denatured form inside cytoplasmic granules (i.e., inclusion bodies) in the host cell. High level expression (i.e., greater than 1 mg recombinant protein/liter of culture) of recombinant proteins often results in the expressed protein being found in inclusion bodies in the host cells. A soluble protein is a protein which is not found in an inclusion body inside the host cell or is found both in the cytoplasm and in inclusion bodies and in this case the protein may be present at high or low levels in the cytoplasm.

The term “hybridization” as used herein, refers to any process by which a strand of nucleic acid binds with a complementary strand through base pairing. Hybridization and the strength of hybridization (i.e., the strength of the association between the nucleic acids) is impacted by such factors as the degree of complementarity between the nucleic acids, stringency of the conditions involved, the temperature of the formed hybrid, and the G:C ratio within the nucleic acids.

As used herein, the term “Tₘ” or “melting temperature” is used in reference to the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ81.5+0.41(% (G+C), when a nucleic acid is in aqueous solution at 1 M NaCl (See e.g., Anderson and Young. Quantitative Filter Hybridization, in Nucleic Acid Hybridization [1985]). Other references include more sophisticated computations which take structural as well as sequence characteristics into account for the calculation of Tₘ.

The term “hybridization complex,” as used herein, refers to a complex formed between two nucleic acid sequences by virtue of the formation of hydrogen binds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., Cₒt or Rₒt analysis) or between one nucleic acid sequence present in solution and another nucleic acid sequence immobilized on a solid support (e.g., membranes, filters, chips, pins or glass slides to which cells have been fixed for in situ hybridization).

The terms “Complement,” “complementary” or “complementarity” as used herein, refer to the natural binding of polynucleotides under permissive salt and temperature conditions by base-pairing. For example, for the sequence “A-G-T” binds to the complementary sequence “T-C-A”. Complementarity between two single-stranded molecules may be partial”, in which only some of the nucleic acids bind, or it may be complete when total complementarity exists between the single stranded molecules. The degree of complementarity between nucleic acid strands has significant effects on the efficiency and strength of hybridization between nucleic acid strands. This is of particular importance in amplification reactions, which depend upon binding between nucleic acids strands.

The term “homology,” as used herein, refers to a degree of complementarity. There may be partial homology or complete homology (i.e., identity). A partially complementary sequence is one that at least partially inhibits an Identical sequence from hybridizing to a target nucleic acid, it is referred to using the functional term “Substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence or probe to the target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence that lacks even a partial degree of complementarity (e.g., less than about 30% identity); in the absence of non-specific binding, the probe will not hybridize to the second non-complementary target sequence.

When used in reference to a single-stranded nucleic acid sequence, the term “substantially homologous” or “homologue” refers to any probe which can hybridize (i.e., it is the complement of) the single-stranded nucleic acid sequence under conditions of low stringency as described.

As known in the art, numerous equivalent conditions may be employed to comprise either low or high stringency conditions. Factors such as the length and nature (DNA, RNA, base composition) of the sequence, nature of the target (DNA, RNA. base composition, presence in solution or immobilization, etc.), and the concentration of the salts and other components (e.g., the presence or absence of formamide, dextran sulfate and/or polyethylene glycol) are considered and the hybridization solution may be varied to generate conditions of either low or high stringency different from, but equivalent to, the above listed conditions.

As used herein the term “stringency” is used in reference to the conditions of temperature, ionic strength, and the presence of other compounds such as organic solvents, under which nucleic acid hybridizations are conducted. With “high stringency” conditions, nucleic acid base pairing will occur only between nucleic acid fragments that have a high frequency of complementary base sequences. Thus, conditions of “weak” or “low” stringency are often required with nucleic acids that are derived from organisms that are genetically diverse, as the frequency of′ complementary sequences is usually less.

Low stringency conditions comprise conditions equivalent to binding or hybridization at 42′° C. in a solution consisting of 5× SSPE (43.8 g/l NaCl, 6.9 g/l NaH₂PO₄.H₂0 and 1.85 g/l EDTA. pH adjusted to 7.4 with NaOH), 0.1% SDS, 5× Denhardt's reagent (50× Denhardt's contains per 500 ml: 5 g Ficoll (Type 400, Pharmacia), 5 g BSA [Fraction V; Sigma]) and 100 μg/ml denatured salmon sperm DNA followed by washing in a solution comprising 5× SSPE, 0.1% SDS at 42° C. when a probe of about 500 nucleotides in length is employed.

The art knows well that numerous equivalent conditions may be employed to comprise low stringency conditions, factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

The term “antisense,” as used herein, refers to nucleotide sequences that are complementary to a specific DNA or RNA sequence. The term “antisense strand” is used in reference to a nucleic acid strand that is complementary to tile “sense” strand. Antisense molecules may be produced by any method, including synthesis by ligating the gene(s) of interest in a reverse orientation to a viral promoter that permits the synthesis of a complementary strand. Once introduced into a cell, the transcribed strand combines with natural sequences produced by the cell to form duplexes. These duplexes block either the further transcription or translation. In this manner mutant phenotypes may be generated. The designation “negative” is sometimes used in reference to the antisense strand, and “positive” is sometimes used in reference to the sense strand.

A gene may produce multiple RNA species that are generated by differential splicing of the primary RNA transcript. cDNAs that are splice variants of the same gene contain regions of sequence identity or complete homology (representing the presence of the same exon or portion of the same exon on both cDNAs) and regions of complete non-identity (for example, representing the presence of exon “A” on cDNA I wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

“Transformation,” as defined herein, describes a process by which exogenous DNA enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection. electroporation, lipofection, and particle bombardment. Such “transformed” cells include stably transformed cells in which the inserted DNA is capable of replication either as an autonomously replicating plasmid or as part of the host chromosome.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microillection, liposome fusion, lipofection, protoplast fusion, retroviral infection, and biolistics. Thus, the term “stable transfection” or “stably transfected” refers to the introduction and integration of foreign DNA into the genome of the transfected cell. The term “stable transfectant” refers to a cell which has stably integrated foreign DNA into the genomic DNA. The term also encompasses cells which transiently express the inserted DNA or RNA for limited periods of time. Thus, the term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

The term “correlates with expression of a polynucleotide,” as used herein, indicates that the detection of the presence of ribonucleic acid that is similar to a particular nucleotide sequence by Northern analysis is indicative of the presence of mRNA encoding a protein in a sample and thereby correlates with expression of the transcript from the polynucleotide encoding the protein.

As used herein, the term “poly-A RNA” refers to RNA molecules having a stretch of adenine nucleotides at the 3′ end. This polyadenine stretch is also referred to as a “poly-A tail”. Eukaryotic mRNA molecules contain poly-A tails and are referred to as poly-A RNA.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro. Cell cultures may comprise insect cells.

As used herein, the term “selectable marker” refers to the use of a gene which encodes an enzymatic activity that confers the ability to grow in medium lacking what would otherwise be an essential nutrient (e.g., the HIS3 gene in yeast cells); in addition, a selectable marker may confer resistance to an antibiotic or drug upon the cell in which the selectable marker is expressed. Selectable markers may be “dominant”, a dominant selectable marker encodes an enzymatic activity which can be detected in any eukaryotic cell line. Examples of dominant selectable markers include the bacterial aminoglycoside 3′ phosphotransferase gene (also referred to as the neo gene) which confers resistance to the drug G418 in mammalian cells, the bacterial hygromycin G phosphotransferase (hyg) gene which confers resistance to the antibiotic hygromycin and the bacterial xanthine-guanine phosphoribosyl transferase gene (also referred to as the gpt gene) that confers the ability to grow in the presence of mycophenotic acid.

Other selectable markers are not dominant in that there use must be in conjunction with a cell line that lacks the relevant enzyme activity. Examples of non-dominant selectable markers include the thymidine kinase (tk) gene which is used in conjunction with tk cell lines, the CAD gene which is used in conjunction with CAD-deficient cells and the mammalian hypoxanthine-guanine phosphoribosyl transferase (hprt) gene which is used in conjunction with hprt, cell lines. A review of the use of selectable markers in mammalian cell lines is provided in Sambrook, J., et al., Molecular Cloning: A Laboratory Manual, 2nd ed., Cold Spring Harbor Laboratory Press, New York (1989) pp. 16.9-16.15.

As used herein, the term “vector” is used in reference to nucleic acid molecules that transfer DNA segment(s) from one cell to another. The term “vehicle” is sometimes used interchangeably with “vector.”

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “in operable combination,” “in operable order,” and “operably linked” as used herein refer to the linkage of nucleic acid sequences in such a manner that a nucleic acid molecule capable of′ directing the transcription of a given gene and/or the synthesis of a desired protein molecule is produced. The term also refers to the linkage of amino acid sequences in such a manner so that a functional protein is produced.

As used herein, the term “amplifiable nucleic acid” is used in reference to nucleic acids that may be “amplified by any amplification method. It is contemplated that “amplifiable nucleic acid” will usually comprise a sample template.”

As used herein, the term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic 'd strand is induced, (i.e., in the presence of nucleotides and an inducing agent such as DNA polymerase and at a suitable temperature and pH). The primer is preferably single stranded for maximum efficiency in amplification but may alternatively be double stranded. If double stranded, the primer is first treated to separate its strands before being used to prepare extension products. The primer will often be an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labelled with any “reporter molecule,” so that is detectable in any detection system, including, but not limited to enzyme (e.g. ELISA, as well as enzyme-based histochemical assays), fluorescent, radioactive, and luminescent systems. It is not intended that the present invention be limited to any particular detection system or label.

As used herein, the term “target” when used in reference to the polymerase chain reaction, refers to the region of nucleic acid bounded by the primers used for polymerase chain reaction. Thus, the “target” is sought to be sorted oat from other nucleic acid sequences. A “segment” is defined as a region of nucleic acid within the target sequence.

As used herein, the term “polymerase chain reaction” (“PCR”) refers to the method of K. B. Mullis U.S. Pat. Nos. 4,683,195, 4,683,202, and 4,965,188, hereby incorporated by reference, which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing. the primers are extended with a polymerase so as to form a new pair of complementary strands. The steps of denaturation, primer annealing and polymerase extension can be repeated many times (i.e., denaturation, annealing and extension constitute one “cycle”, there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection, incorporation ³²P-labeled deoxynucleotide triphosphates, such as DCTP or DATP, into the amplified segment). In addition to genomic DNA, any oligonucleotide sequence can be amplified with the appropriate set of primer molecules. In particular the amplified segments created by the PCR process itself are, themselves, efficient templates for subsequent PCR amplifications.

“Amplification” is a special case of nucleic acid replication involving template specificity. It is to be contrasted with nonspecific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under conditions they are used, will process only specific sequences of nucleic acid in a heterogeneous mixture of nucleic acid. For example,, in the case of Qβ replicase, MDV-1 RNA is the specific template for the replicase (D. L. Kacian et al, Proc. Natl. Acad. Sci. USA 69:3038 [1972]). Other nucleic acid will not be replicated by this amplification enzyme. Similarly, in the case of T7 RNA polymerase, this amplification enzyme has a stringent specificity for its own promoters (M. Chamberlin et al Nature 228:227 [1970]). In the case of T4 DNA ligase, the enzyme will not ligate the two oligonucleotides where there is a mismatch between the oligonucleotide substrate and the template at the ligation junction (D. Y. Wu and R. B. Wallace, Genomics 4:560 [1989]). Finally, Taq and Pfu polymerases by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press [1989]).

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “amplification reagents” refers to those reagents (deoxyribonucleotide triphosphates, buffer, etc.), needed for amplification except for primers. nucleic acid template and the amplification enzyme. Typically, amplification reagents along with other reaction components are placed and contained in a reaction vessel (test tube, microwell, etc.).

As used herein, the term “restriction endonucleases” and “restriction enzymes” refer to bacterial enzymes, each of which cut double-stranded DNA at or near a specific nucleotide sequence.

As used herein, the term “recombinant DNA molecule” as used herein refers to a DNA molecule which is comprised of segments of DNA joined together by means of molecular biological techniques.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end oligonucleotides referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends. In either a linear or circular DNA molecule, discrete elements arc referred to as being “upstream” or 5′ of the “downstream” or 3′ elements. This terminology reflects the fact that transcription proceeds in a 5′ to 3′ fashion along the DNA strand. The promoter and enhancer elements which direct transcription of′ a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the term “a gene encoding,” “nucleic acid molecule encoding,” “DNA sequence encoding,” and “DNA encoding” means a nucleic acid sequence comprising the coding region of a gene or in other words the nucleic acid sequence which encodes a gene product, such as a protein. In the case of DNA, for example, the terms refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

The coding region may be present in either a CDNA., genomic DNA or RNA form. When present in a DNA form, the oligonucleotide may be single-stranded (i.e., the sense strand) or double-stranded. Suitable control elements such as enhancers/promoters, splice.junctions, polyadenylation signals, etc. may be placed in close proximity to the coding region of the gene if needed to permit propei-initiation of transcription and/or correct processing of the primary RNA transcript. Alternatively, the coding region utilized in the expression vectors of the present invention may contain endogenous enhancers/promoters, splice junctions, intervening sequences, polyadenylation signals. etc. or a combination of both endogenous and exogenous control elements.

As used herein, the term “regulatory element” refers to a genetic element which controls some aspect of the expression of nucleic acid sequences. For example, a promoter is a regulatory element which facilitates the initiation of transcription of an operably linked coding region. Other regulatory elements are splicing signals, polyadenylation signals, termination signals, etc. (defined in infra).

Transcriptional control signals in eukaryotes comprise “promoter” and “enhancer” elements. Promoters and enhancers consist of short arrays of DNA sequences that interact specifically with cellular proteins involved in transcription (T. Maniatis et al., Science 236:1237 [1987]). Promoter and enhancer elements have been isolated from a variety of eukaryotic sources including genes in yeast, insect and mammalian cells and viruses (analogous control elements, i.e., promoters, are also found in prokaryotcs). The selection of a particular promoter and enhancer depends on what cell type is to be used to express the protein of interest. Some eukaryotic promoters and enhancers have a broad host range while others are functional in a limited subset of cell types (for review see, S. D. Voss et al, Trends Biochem. Sci., 11:287 [1986]; and T. Maniatis et al, supra). For example, the SV40 early gene enhancer is very active in a wide variety of cell types from many mammalian species, and has been widely used for the expression of proteins in mammalian cells (R. Dijkema et al, EMBO J. 4:761 [19851). Two other examples of promoter/enhancer elements active in a broad range of mammalian cell types are those from the human elongation fictor Iα gene (T. Uetsuki et al, J. Biol. Chem., 264:5791 [1989] D. W. Kim et al., Gene 91:217 [1990] and S. Mizushima Lind S. Nagata, Nuc. Acids Res., 18:5322 [1990]), and the long terminal repeats of the Rous sarcoma virus (C. M. Gorman et al., Proc. Natl. Acad. Sci. USA 79:6777 [1982]), and the human cytomegalovirus (M. Boshart et al., Cell 41:521 [1985]).

As used herein, the term “promoter/enhancer” denotes a segment of DNA which contains sequences capable of providing both promoter and enhancer functions (i.e., the functions provided by a promoter element and an enhancer element, as discussed above). For example, the long terminal repeats of retroviruses contain both promoter and enhancer functions. The enhancer/promoter may be “endogenous,” “exogenous,” or “heterologous.” An “endogenous” enhancer/promoter is one which is naturally linked with a given gene in the genone. An “exogenous” or “heterologous” enhancer/promoter is one which is placed in juxtaposition to a gene by means of genetic manipulation (i.e., molecular biological techniques), such that transcription of that gene is directed by the linked enhancer/promoter.

The presence of “splicing signals” on an expression vector often results in higher levels of expression of the recombinant transcript. Splicing signals mediate the removal of introns from the primary RNA transcript and consist of a splice donor and acceptor site (See e.g., J. Sambrook et al. Molecular Cloning—A Laboratory Manual, 2nd ed., Cold Spring Harbor Laboratory Press, New York [1989], pp. 16.7-16.8). A commonly used splice donor and acceptor site is the splice junction from the 16S RNA of SV40.

Efficient expression of recombinant DNA sequences in eukaryotic cells requires expression of signals directing the efficient termination and polyadenylation of the resulting transcript. Transcription termination signals are generally found downstream of the polyadenylation signal and are a few hundred nucleotides in length. The term “poly A site” or “poly A sequence,” as used herein, denotes a DNA sequence that directs both the termination and polyadenylation of the nascent RNA transcript. Efficient polyadenylation of the recombinant transcript is desirable, as transcripts lacking a poly A tall are unstable and are rapidly degraded. The poly A signal utilized in an expression vector may be “heterologous” or “endogenous.” An endogenous poly A signal is one that is found at the 3′ end of the coding region of a given gene in the genome. An heterologous poly A signal is one which is isolated from one gene and placed 3′ to another gene. A commonly used heterologous poly A signal is the SV40 poly A signal. The SV40 poly A signal is contained on a 237 bp BamHI/Bcl restriction fragment, and directs both termination and polyadenylation (S. Sambrook, supra, at 16.6-16.7).

Eukaryotic expression vectors may also contain “viral replicons,” or “viral origins of replication.” Viral replicons are viral DNA sequences which allow for the extrachromosomal replication of a vector in a host cell expressing the appropriate replication factors.

The term “Southern blot” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (See e.g., J. Sambrook et al., supra at pp 9.31-9-58).

The term “Northern blot” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (See e.g., Sambrook et al., supra at pp. 7) 9-7.52).

The term “isolated” when used in relation to a nucleic acid, as in “an isolated oligonucleotide” refers to a nucleic acid sequence that is identified and separated from at least one containment nucleic acid with which it is ordinarily associated in its natural source. Isolated nucleic acid is such present in a form or setting that is different from that in which it is found in nature (e.g., in an expression vector). In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs which encode a multitude of proteins. However, isolated nucleic acid encoding a mammalian protein includes, by way of example, such nucleic acid in cells ordinarily expressing, a protein where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide will contain at a minimum the sense or coding strand (i.e., the oligonucleotide may single-stranded), but may contain both the sense and anti-sense strands (i.e., the oligonucleotide may be double-stranded).

As used herein, the term “immunogen” refers to a substance, compound, molecule, or other moiety which stimulates the production of an immune response. The term “antigen” refers to a substance, compound, molecule, or other mioety that is capable of reacting with products of the immune response. For example, proteins may be used as immunogens to elicit an immune response in an animal to produce antibodies directed against the subunit used as an immunogen. The subunit may then be used as an antigen in an assay to detect the presence of antibodies against the protein in the serum of the immunized animal. It is not intended that the present invention be limited to antigens or immunogens consisting solely of one protein. Nor is it intended that the present invention be limited to any particular antigens or immunogens.

The term “antigenic determinant,” as used herein, refers to that portion of a molecule (i.e., an antigen) that makes contact with a particular antibody (i.e., an epitope). When a protein or fragment of a protein is used to immunize a host animal (e.g., an “immunocompetent” animal with “immunocompetent cells”), numerous regions of the protein may induce the production of antibodies which bind specifically to a given region or three-dimensional structure on the protein; these regions or structures are referred to as antigenic determinants. An antigenic determinant may compete with the intact antigen (i.e., the immunogen used to elicit the immune response) for binding to an antibody.

The terms “specific binding” or “specifically binding,” as used herein, in reference to the interaction of an antibody and a protein or peptide, mean that the interaction is dependent upon the presence of a particular structure (i.e., the antigenic determinant or epitope) on the protein; in other words, the antibody is recognizing and binding to a specific protein structure rather than to proteins in general. For example, if an antibody is specific for epitope “A”, the presence of a protein containing epitope A (or free, unlabeled A) in a reaction containing labeled “A” and the antibody will reduce the amount of labeled A bound to the antibody.

As used herein, the term “antibody” (or “immunoglobulin”), refers to intact molecules as well as fragments thereof, such as Fa, F(ab′)₂, and Fᵥ, which are capable of binding the epitopic determinant. Antibodies that bind polypeptides can be prepared using intact polypeptides or fragments containing small peptides of interest as the immunizing antigen. The polypeptide or peptide used to immunize an animal can be derived from the transition of RNA or synthesized chemically, and can be conjugated to a carrier protein, if desired. Commonly used carriers that are chemically coupled to peptides include bovine serum albumin and thyroglobulin. The coupled peptide is then used to immunize the animal (e.g., a mouse, a rat, or a rabbit).

The present invention encompasses polyclonal, as well as monoclonal antibodies. The antibodies used in the methods of the invention may be prepared using various immunogens. The immunogen may be a human protein or subunit (e.g., any of the amino acid sequences set forth herein used as an immunogen) to generate antibodies that recognize human proteins. Such antibodies include, but are not limited to polyclonal, monoclonal, chimeric, single chain,, Fab fragments, and an Fab expression library.

Various procedures known in the art may be used for the production of polyclonal antibodies to proteins and subunits. For the production of antibodies, various host animals can be immunized by injection with the peptide corresponding to the protein epitope of interest, including but not limited to rabbits, mice, rats, sheep, goats, etc. In a preferred embodiment, the peptide is conjugated to an immunogenic carrier (e.g., diphtheria toxoid, bovine serum albumin (BSA). or keyhole limpet hemocyanin [KLH]). Various adjuvants may be used to increase tile immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum.

For preparation of monoclonal antibodies directed toward proteins, any technique that provides for the production of antibody molecules by continuous cell lines in culture may be used (See e.g., Harlow and Line, Antibodies: A Laboratory Manual, Cold Spring Harbor Laboratory Press, Cold Spring Harbor, N.Y.). These include but are not limited to the hybridoma technique originally developed by Kohler and Milstein (Kohier and Milstein, Nature 256:495-497 [1975]), as well as the trioma technique, the human B-cell hybridoma technique (See e.g., Kozbor et al., Immunol. Today 4:72 [1983]), and the EBV-hybridoma technique to produce human monoclonal antibodies (Cole et al., in Monoclonal Antibodies and Cancer Therapy, Alan R. Liss, Inc., pp. 77-96 [1985]).

Monoclonal antibodies can be produced in germ-free animals utilizing recent technology (See e.g., PCT/US90/02545). According to the invention, human antibodies may be used and can be obtained by using human hybridomas (Cote et al., Proc. Natl. Acad. Sci. U.S.A. 80:2026-2030 [198′)]) or by transforming human B cells with EBV virus in vitro (Cole et al, in Monoclonal Antibodies and Cancer Therapy, Alan R. Liss, pp. 77-96 [1985]).

According to the invention, techniques described for the production of single chain antibodies (U.S. Pat. No. 4,946,778, herein incorporated by reference) can be adapted to produce protein-specific single chain antibodies. The techniques described for the construction of Fab expression libraries (Huse et al., Science 246:1275-1281 [1989]) allow rapid and easy identification of monoclonal Fab fragments with the desired specificity for proteins.

Antibody fragments which contain the idiotype (antigen binding region) of the antibody molecule can be generated by known techniques. For example, such fragments include but are not limited to: the F(ab′)2 fragment which can be produced by pepsin digestion of the antibody molecule; the Fab′ fragments which can be generated by reducing the disulfide bridges of the F(ab′)2 fragment, and the Fab fragments which can be generated by treating the antibody molecule with papain and a reducing agent.

In the production of antibodies, screening for the desired antibody can be accomplished by techniques known in the art (e.g., radioimmunoassay, ELISA [enzyme-linked immunosorbant assay], 24 “sandwich” immunoassays, immunoradiometric assays, gel diffusion precipitin reactions, immunodiffusion assays, in situ immunoassays [using colloidal gold, enzyme or radioisotope labels, for example], Western Blots, precipitation reactions, agglutination assays (e.g., gel agglutination assays, hemagglutination assays, etc.), complement fixation assays, immunofluorescence assays, protein A assays, and immunoetectrophoresis assays, etc.

As used herein the term “immunogenically-effective amount” refers to that amount of an immunogen required to invoke the production of protective levels of antibodies in a host upon vaccination.

As used herein, the term “reporter reagent” or “reporter molecule” is used in reference to compounds which are capable of detecting the presence of antibody bound to antigen. For example, a reporter reagent may be a calorimetric substance which is attached to an enzymatic substrate. Upon binding of antibody and antigen, the enzyme acts on its substrate and causes the production of a color. Other reporter reagents include, but are not limited to fluorogenic and radioactive compounds or molecules.

As used herein the term “signal” is used in reference to the production of a sign that a reaction has occurred, for example, binding of antibody to antigen. It is contemplated that signals in the form of radioactivity, flurogenic reactions, and enzymatic reactions will be used with the present invention. The signal may be assessed quantitatively as well as qualitatively.

As used herein the term “regulatory factors” refers to any factors (e.g., proteins, enzymes, peptides, small molecules, and nucleic acids) involved in the regulation of signaling pathways. For example, such factors include, but are not limited to, proteins, IKBS, IKKS, and agonists, antagonists, and cofactors that interact with these factors. It is contemplated that the regulatory factors can either directly or indirectly (e.g., through other factors) bind to a target of interest.

Membrane Steroid Receptors From Spotted Seatrout

A membrane progestogen receptor was solubilized from spotted seatrout (Cynoscion nebulosus) ovary using a detergent, triton X-100, and purified on a DEAE column. Monoclonal antibodies to the partial purified progestin membrane receptor were then generated in mice. Positive monoclonal antibodies were selected by analyses of ELISA and Western blot and subsequently by their ability to bind to the solubilized progestogen receptor in 96-well plates using a hormone receptor capture protocol developed in the present invention.

Antibodies showing positive results were considered to recognize portions of the membrane progestin receptor and therefore suitable for further cDNA library screening. The cloned membrane progestin receptor was expressed in E. coli and the binding of the recombinant protein to steroids was analyzed by a radiolabeled receptor assay.

Membrane fractions of spotted seatrout ovarian tissue were obtained following procedures described previously (Patino and Thomas, 1990) with a few modifications. Twenty grams of ovarian tissue were homogenized in 120 ml ice-cold homogenization-assay (HA) buffer (Hepes, 25 mM; NaCl, 10 mM; dithioerythritol, 1 mM) using a Polytron Tissuemizer at setting 7 for 45 seconds (Tekmar, Cincinnati, Ohio). The ovarian homogenate was centrifuged for 20 minutes at 20,000 g. The membrane pellet was resuspended in 120 ml ice HA buffer using the Tissuemizer at setting 7 for 20 seconds and centrifuged for 10 minutes at 1000 g. The supernatant was collected and centrifuged for 1 hour at 160,000 g. The resulting pellet was resuspended in HA buffer and centrifuged again twice for 1 hour at 160,000 g to remove cytosolic proteins. The final pellet (Pellet II) was stored at −70° C. for up to 6 months prior to solubilization or analysis of receptor binding.

Various solubilization procedures for ovarian membrane proteins were evaluated. Pellet II was resuspended in 4 volumes HA buffer containing different concentrations of digitonin, CHAPS and Triton X-100 (2.4 mM to 120 mM). The mixture was homogenized using a Polytron tissuemizer at setting 7 for 15 seconds and the mixture was stirred for periods ranging from 15 minutes to 4 hour to solubilize the membrane proteins. The mixture was subsequently centrifuged and the supernatant containing the solubilized proteins was used in subsequent analyses. Thereafter, the detergents were removed by stirring the supernatants with polystyrene adsorbents (2:1 v/w; BioRad SM-2) for 2 hour. The polystyrene adsorbents were removed by filtration (G-8 filter, Fisher) and the final supernatant was stored at −70° C. until further analysis.

20β-S receptor binding in intact membrane preparations was assayed using a filtration method to separate bound from free steroid as described by Patino and Thomas (1990). Each sample was assayed in duplicate with radiolabeled ligand in all tubes and 1000-fold excess unlabeled ligand in the nonspecific binding tubes. Radioactive ligand was dissolved in HA buffer, and 250 μl aliquots of this solution were pipetted into test tubes (final concentration 5 nM for 20β-S) with or without the nitrogen-evaporated unlabeled ligand. A-250 μl aliquot of plasma membrane was added to each tube, and the reaction mixture was incubated on ice for 30 minutes. Receptor-bound steroid was separated from free steroid by adding 400 μl of the reaction mixture to a glass microfiber filter (Whatman, GF/B) attached to a vacuum source, followed by a rinse with 12 ml of HA buffer.

20β-S receptor binding in solubilized membrane preparations was assayed using dextran-coated charcoal (DCC) to separate bound from steroid. The incubation conditions were similar to those used for the intact membrane preparation with the exception that 100-fold excess unlabeled 20β-S was used to determine non-specific binding. The incubation was terminated by adding 1-ml ice-cold DCC suspension (0.1 g dextran T-70 and 1 g charcoal per 100 ml HA buffer). The tubes were vortexed and the reaction mixture was incubated on ice for 5 minutes to absorb free steroid followed by centrifugation at 3200 g for 10 minutes at 4° C. The radioactivities of the filters or supernatants were measured using Beckman LS 6000SC Scintillation Counter (Beckman Instruments Inc., Houston, Tex.).

Saturation kinetics of [³H]20β-S binding to the solubilized membrane receptor was conducted with six radiolabeled 20β-S concentrations (range 0.25-10 nM) in the absence (total binding) or presence of 100-fold excess cold steroid (non-specific binding). The dissociation constant (Kd) and receptor concentration (Bₘₐₓ) were calculated from Scatchard plots. One point assays were conducted with 5 nM radiolabeled 20β-S with or without 100-fold excess unlabeled steroid.

The rate of 20β-S association to the solubilized receptor was determined by measuring the specific binding of [³H]20β-S after-incubation periods of 30 seconds to 24 hours. The rate of dissociation was determined by first allowing samples to equilibrate with tracer for 30 minutes before the addition of 100-fold excess unlabeled 20β-S and incubation for periods of 30 seconds to 24 hours.

Solubilized proteins were separated on a DEAE (Pharmacia) column (40×1 cm) equilibrated with 25 mM Tris buffer (pH 7.8). The proteins were eluted with 50 ml 25 mM Tris and 50 ml Tris buffer containing 0.1 M or 0.5 M NaCl. Fractions eluting from each step were pooled and further purified and concentrated to 1.2 ml by a molecular weight cutoff centrifuge concentrator (Amico, Centriprep 10 concentrator, MWCF 10,000). Aliquots of purified each fraction were analyzed by SDS-PAGE.

Two BALB/c mice were immunized with purified proteins (100 μg/mice) and complete adjuvant (1:1 v/v, total 150 μl/mouse). Booster injections were given at 35 days and 56 days following initiation of immunization. Splenocytes were fused on day 60. Positive antibodies were selected by screening with ELISA and Western blotting.

Positive antibodies to the 20β-S receptor were selected using a double-antibody-receptor capture assay developed by the present inventors. All the reactions were carried out in a humidified chamber at 4° C. except where indicated. Each well of a 96-well culture plate (Nunc, ImmunoBreak Module) was coated with a second antibody by adding 200 μl coating buffer (0.05 M carbonate buffer, 0.05% NaN₃, pH 8.4) containing 15 μg/ml goat IgG against mouse IgG FC fragments (Sigma) and incubated for 2 days at 4° C. The plates were washed three times with 0.85% NaCl at end of the incubation period to remove excess unbound antibody. The plates were then incubated overnight with 300 μl blocking solution (0.05 M PBS, 0.1% BSA, 3% sucrose, 0.005% thimerosal, pH 7.0) to saturate the nonspecific binding sites.

After removing the buffer and blotting the plates on paper towels, 250-μl aliquots of the solubilized membrane proteins and 50-μl of mouse IgG culture supernatants were added to each well and the mixture was incubated for overnight with slight agitation in order for goat IgG-mouse IgG-membrane protein to form a stable complex. The next day, extra membrane proteins were stripped off the wells by draining the reaction mixture and washing them three times with HA buffer.

Thereafter, all the wells received 100 μl of labeled 20β-S (final concentration 5 nM) with additional 100-μl HA buffer for total binding or 100 μl of 1000-fold excess cold 20β-S for non-specific binding. The plates were incubated on ice for 30 minutes. The aqueous mixture was removed and each well was washed twice with HA buffer. Each individual well was broken off and placed in a 7-ml scintillation vial containing 5-ml scintillation cocktail and counted for radioactivity in a Beckman LS 6000SC Scintillation Counter (Beckman Instruments Inc., Houston, Tex.). Positive monoclonal antibodies were identified based on specific binding to steroid.

Total RNA was extracted from seatrout ovarian tissue (2.1 g) using 30-ml TRIZOL extraction reagent based on the acid guanidinium thiocyanate-phenol-chloroform extraction method (AGPC method). The mRNA was purified from total RNA by magnetic-oligo (dT) particles using Straight A's mRNA Isolation System (Novagen). First- and second-strand cDNAs were synthesized from the polyadenylated mRNA using a TimeSaver cDNA Synthesis Kit (Pharmacia). Synthesized double-strand cDNAs were linked to adaptors containing an EcoRl linker and an internal Notl site. A seatrout ovarian cDNA library was constructed following ligation of synthesized cDNAs into EcoRl sites of λZAP II vectors and packaging of ligated λZAP vectors (Stratagene).

Expressed fusion proteins from a total of 300,000 phages were screened employing the previously-obtained mouse monoclonal antibodies and goat anti-mouse picoblue immunoscreening kit (Stratagene). The positive clones were further subjected to two additional rounds of screening prior to excision of pBluescript SK plasmid vector from λZAP II phage. The plasmids were purified from selected positive E. coli clones for further analysis.

Sequencing was performed for both strands of the selected cDNA clones using automated DNA sequencers (model 377 or 310, Perkin-Elmer Applied Biosystems). Computer sequence analysis was carried out using the Wisconsin Package GCG programs (Version 9.1, Genetics Computer Group, Madison, Wis., U.S.A.) on a UNIX platform (Sun 5.3, SUN Microsystems, Calif., U.S.A.) and MacVector (Version 6.1, Oxford). A homologous search of sequences was carried out using Fasta (GCG) and BLAST (NCBI, National Center for Biotechnology Information).

The coding region of the steroid membrane receptor was amplified by PCR from a full-length cDNA plasmid clone. Polymerase chain reaction (PCR) was carried out in 100 μl aliquots containing 50 mM KCl, 10 mM Tris (pH 8.3), 1.5 mM MgCl₂, 0.2 mM DNTP, a set of 5′ and 3′ primers (1 μM each) and 2.5 units of Taq DNA polymerase mix. After an initial 2-minutes denaturation at 94° C., the PCR cycle reaction was repeated 5 times with denaturation at 94° C. for 1 minutes, annealing at 50° C. for 1 minutes and polymerization at 72° C. for 2 minutes. Subsequently, 25 cycles of PCR reaction were carried out under the same conditions except annealing which was conducted at 55° C.

The PCR products were purified by electrophoresis using low-melting gel and a QIAquick Gel Extraction Kit. The purified PCR product was ligated into a pET expression vector. The pET vector was transformed into BL21 (DE3) E. coli cells. The positive clones were purified and sequenced. The plasmid without an insert was used as control. Recombinant protein was induced with IPTG at 25° C. and soluble recombinant proteins were extracted and analyzed.

Total RNA was isolated from various tissues (1 g) using 10-20 ml TRIZOL extraction reagent (Life Technologies). Ovary, testis, brain, pituitary, liver, kidney, muscle, intestine and gill tissues were immersed immediately in TRIZOL reagent following excision from a mature spotted seatrout. The expression of the receptor in tissues was examined by Northern hybridization using total RNA and a Northern Max Kit (Ambion, Austin, Tex.). The RNA was denatured at 65° C. for 15 minutes in formaldehyde loading buffer prior to electrophoresis on 1.2% agarose gel containing formaldehyde. The RNA was then transferred to a positively-charged nylon membrane within 90 minutes using a downward transfer Turboblotter. The transferred mRNA was then cross-linked to the membrane by baking at 80° C. for 15 minutes.

Probes of the receptor were prepared by digesting purified plasmids containing cDNA inserts with appropriate enzymes, and the desired fragments were purified by electrophoresis using a low-melting gel and a QlAquick Gel Extraction Kit. Thereafter, 20-25 ng of DNA were randomly labeled with Pharmacia Rediprime Kit and Amersham Redivue [α-³²P]dCTP, and the labeled probe was purified using a Pharmacia ProbeQuant G-50 micro spin column. The incorporation rate of [α-³²P]CTP ranged from 50-80% and specific activity of probes ranged from 1.2-1.6×10⁹ cpm/μg.

The membrane was hybridized with a labeled probe at a final concentration of approximately 5×10⁶ cpm/ml at 42° C. overnight. Non-specific binding was stripped off the membrane by washing twice with 20 ml low-stringency buffer for 5 minutes at 42° C. and twice with 20 ml high-stringency buffer twice for 15 minutes at 42° C. The hybridized membrane was exposed to a Kodak Biomax MS film for 3 hours several days prior to development.

Gene Family Identification

Human genome sequences similar to the fish progestin membrane receptor were obtained by BLAST searching using the deduced amino acid sequence of seatrout membrane progestin receptor in the htgs database of the unfinished human genome sequence. EST cDNA clones of membrane steroid receptors were identified by BLAST searching of the human genome sequence and partial cDNA sequence found in the EST database. These clones were subsequently obtained or purchased from National Institute of Infectious Diseases (Tokyo, Japan), Royal Veterinary and Agricultural University (Denmark), Oakland Children Hospital (Oakland, Calif.) and Research Genetics (Huntsville, Ala.).

Full cDNA sequences of the clones was determined for both strands using the primer-walking method and automated DNA sequencers (model 310, Perkin-Elmer Applied Biosystems). An additional full-length human cDNA clone was found in no database of GenBank and obtained from Dr. Sugano at Tokyo University Japan. Recombinant protein expression and receptor binding analyses of two fun-length human cDNA clones were performed as described in the previous sections. Human multiple tissue expression array and multiple tissue Northern blots were purchased from Clonetech. Northern hybridization was performed following the manufacturer's instruction.

Solubilization, purification and antibody generation for membrane progestin receptor from ovaries of seatrout.

Progestin (20β-S) binding was diminished or completely disappeared from the membrane fraction following solubilization treatment with several detergents including digitonin, CHAPS or Triton X-100 at various concentrations (results only shown with 12 mM Triton X-100) and appeared in the soluble fraction (FIG. 1).

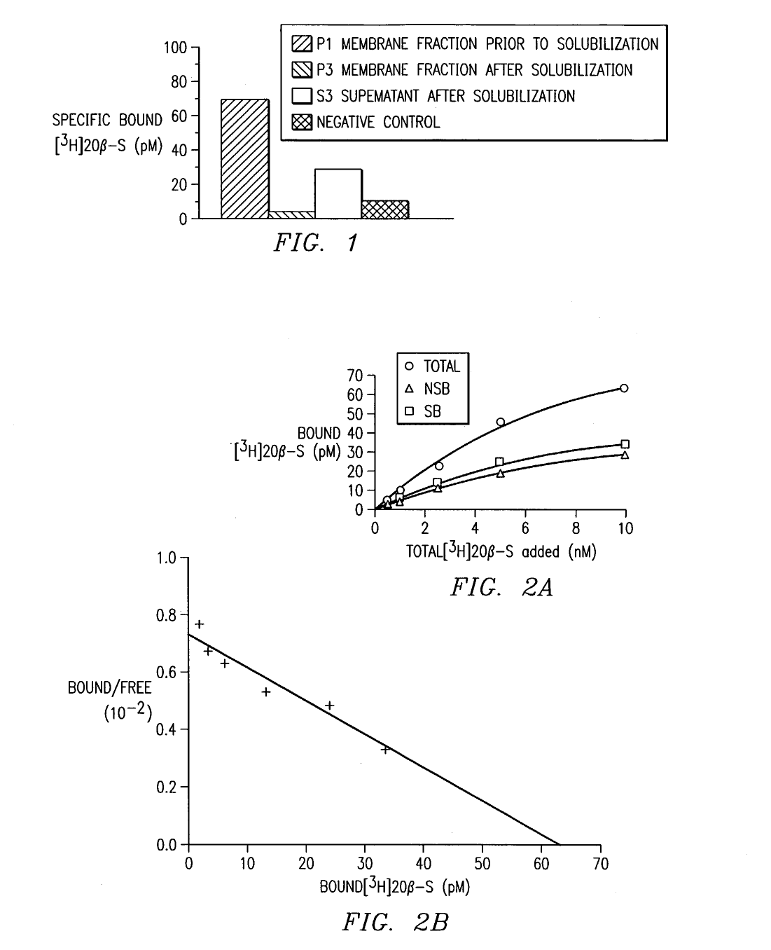

Saturation analysis showed the presence of saturable progestin (2062 -S) binding in the solubilized ovarian membrane proteins (insert). Saturation was achieved with approximately 10 nM 20β-S. Scatchard analysis indicated the presence of a single class of high affinity (Kd=8.7 nM) low capacity (Bmax=0.204 pmol g⁻¹ ovary) binding sites in the ovarian plasma membrane (FIG. 2).

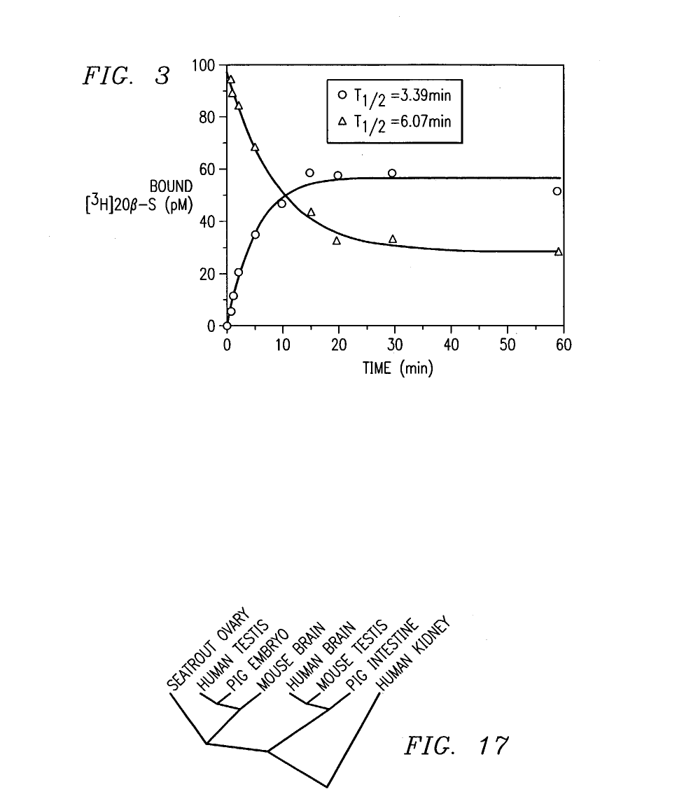

Equilibrium studies indicated that binding reached saturation rapidly (within 15 minutes, T_{1/2}=3.4 minutes) and that specific binding remained constant throughout the 24-hour period tested (data shown only for time points between 30 see and 60 minutes). The rate of dissociation (T_{1/2}=6.1 minutes) was slightly slower than the rate of association with full displacement occurring within 20 minutes (FIG. 3).

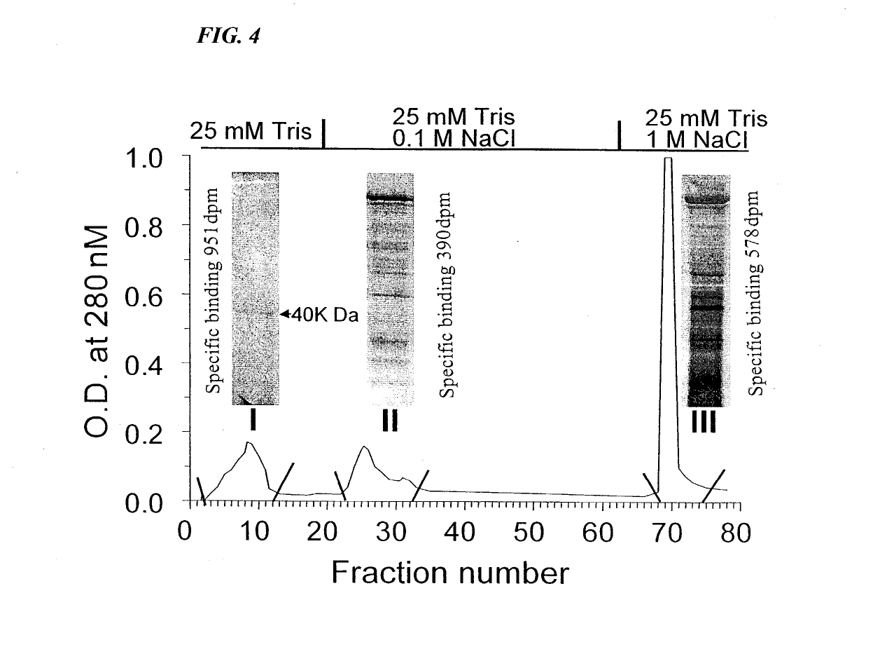

Solubilized proteins were eluted and pooled as shown in FIG. 4. Each pooled fraction was further purified and concentrated to 2 mg protein/ml with a 10,000 molecular weight cutoff centrifuge concentrator. The proteins from the first fraction (flow through) showed the greatest receptor binding. SDS-page analyses showed a major band with a molecular weight around 40K Da (FIG. 4, insert 1). This purified first fraction was used to immunize mice to generate monoclonal antibodies (FIG. 4).

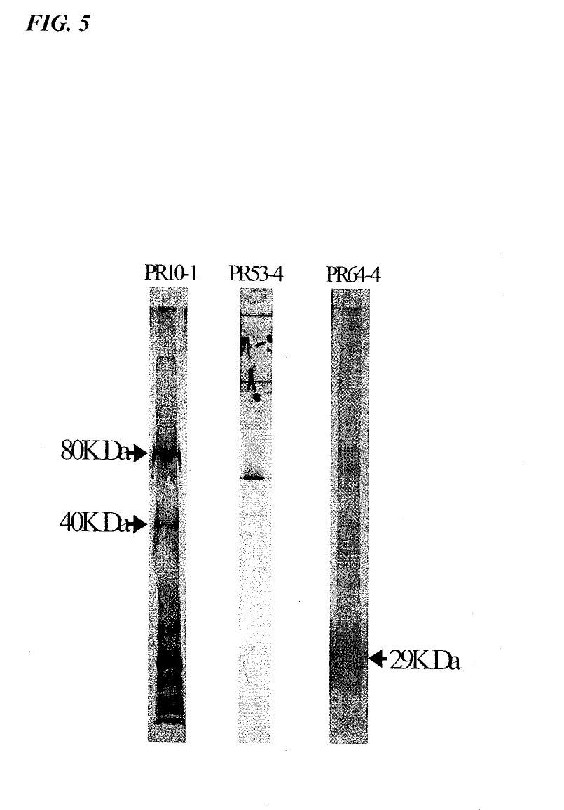

Seventeen partially cloned positive antibodies were first selected by screening with ELISA based on positive immunoreactions with solubilized proteins. Eight of seventeen antibodies were further selected based on positive immunoreactive bands with DEAE purified proteins ranging from 25,000-100,000 Da on Western blots. The selected antibodies were further cloned and screened with a double antibody capture assay. Three positive monoclonal antibodies (PR 10-1, PR 53-4, PR 64-4) were finally identified using the capture assay. These antibodies recognized various ovarian protein bands ranging from 29-80 Kda on western blots (FIG. 5).

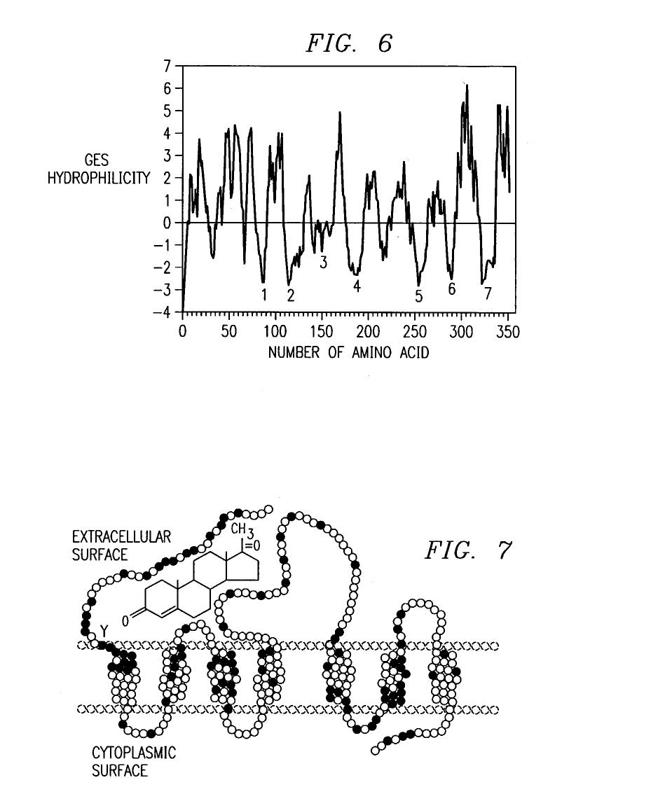

The three selected monoclonal antibodies were used for screening a seatrout ovarian expression cDNA library for a steroid membrane receptor. Nine clones were isolated and sequenced. Out of those 9 clones, a cDNA fragment (1.4 Kb) was completely sequenced and appeared to be a novel gene of SEQ ID NO: 1 (GenBank accession AF262028), not related to any previously characterized vertebrate gene. The deduced amino acid sequence of this gene (SEQ ID NO: 2) showed 7 possible transmembrane domains by hydrophilicity analysis, which is characteristic of G-protein-coupled membrane receptors (FIGS. 6, 7).

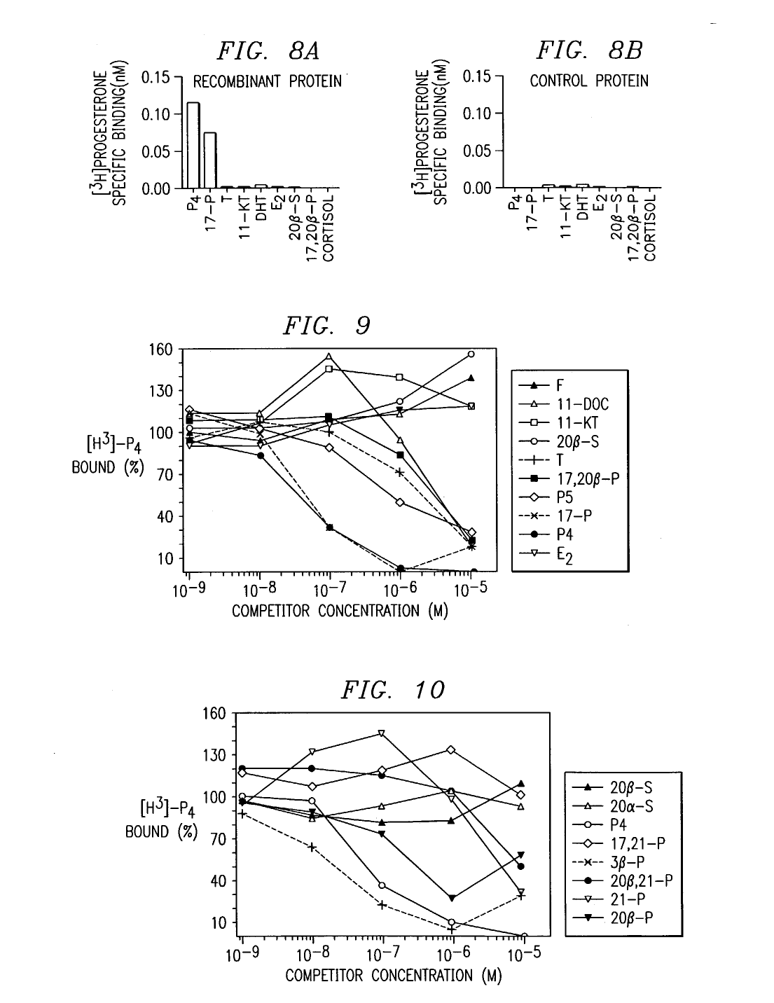

Initially, steroid specificity of the recombinant receptor was determined from single-point competition assays using 10 nM ³H-steroid in the absence (total binding) or presence of 100-fold excess non-radioactive steroid (nonspecific binding, FIG. 8). Highest specific binding was obtained for ³H-progesterone (P4). The receptor also showed high affinity for ³H-17-hydroxyprogesterone (17-P). No binding of P₄ and 17-P to the control proteins was observed (FIG. 7). The receptor binding was steroid specific and no specific binding was observed with androgens including testosterone (T), 11-ketotestosterone (11-KT) and 5α-dihydrotestosterone (DHT). In addition, 17,20β,21-trihydroxy-4-pregnen-3-one (20β-S) and 17,20β-dihydroxy-4-pregnen-3-one (17,20β-P) did not show any specific binding.

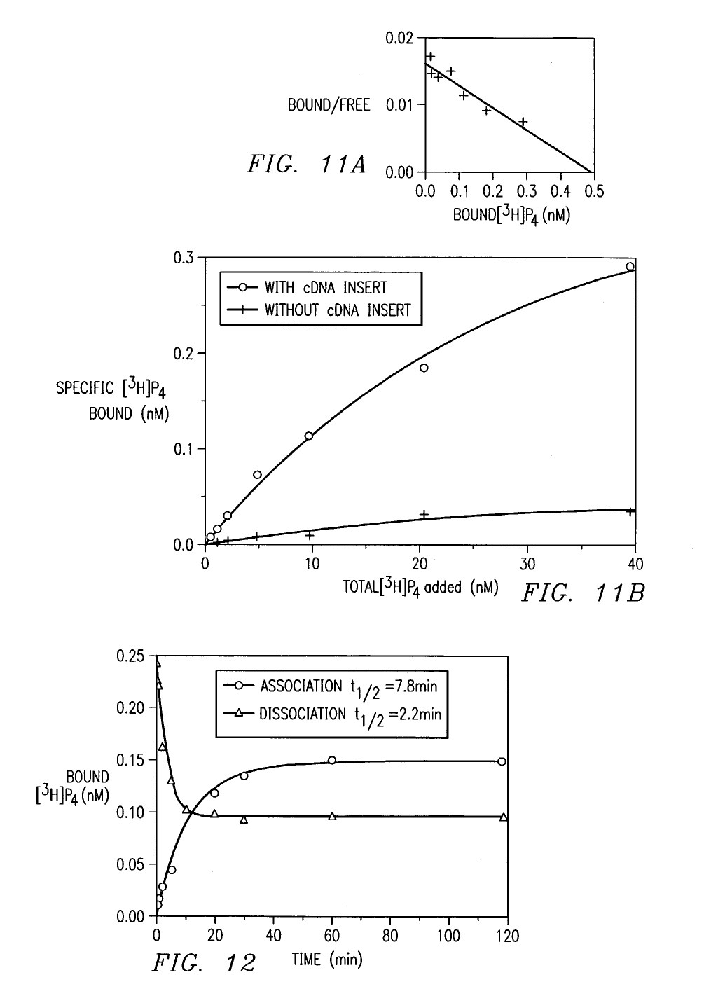

Full competition curves were generated with 10 nM 3H-progesterone (total binding) or in combination with 1 nM-10 μM of various steroids (FIG. 8). 17-P was equipotent with P4 in displacing ³H-progesterone (FIGS. 9, 10). The binding was very specific for 21 carbon steroids lacking a functional group at the 11 position (FIG. 9). The presence of single or multiple hydroxyl (OH) groups at several positions on the side chain of the 21 carbon steroids (pregnenes), however, decreased displacement of ³H-progesterone, whereas an OH at the 3β position increased ³H-progesterone displacement (FIG. 10).

Saturation kinetics of H³-progesterone binding to the recombinant receptor were conducted with 7 progesterone concentrations (range 0.5-40 nM) in the absence (total binding) or presence of 1 μM excess cold steroid (nonspecific binding, FIG. 11). Scatchard analyses confirmed the presence of a single class of high affinity (Kd=30 nM, FIG. 11 insert), saturable binding sites (Bmax=0.49 nM). The E. coli control proteins showed no specific binding to H³-progesterone (FIG. 11).

Association of the ligand for the steroid membrane receptor was rapid with a t_{1/2} association 7.8 minutes. The specific binding dissociated quickly with a t_{1/2} dissociation of 2.2 minutes (FIG. 12).

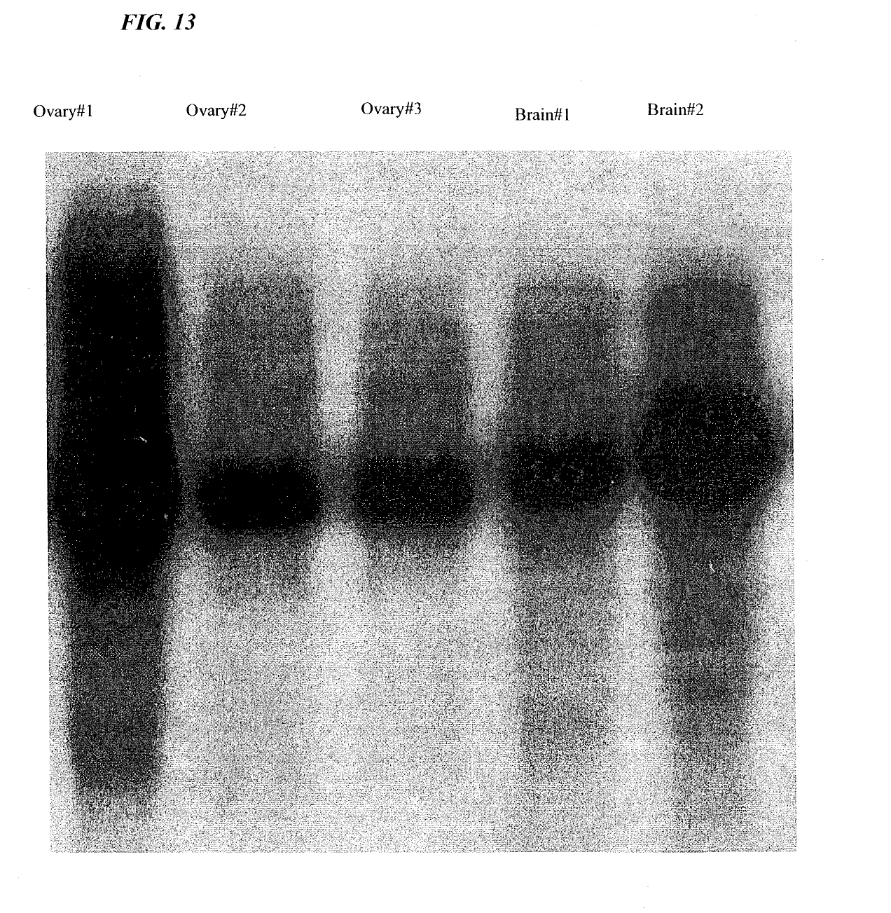

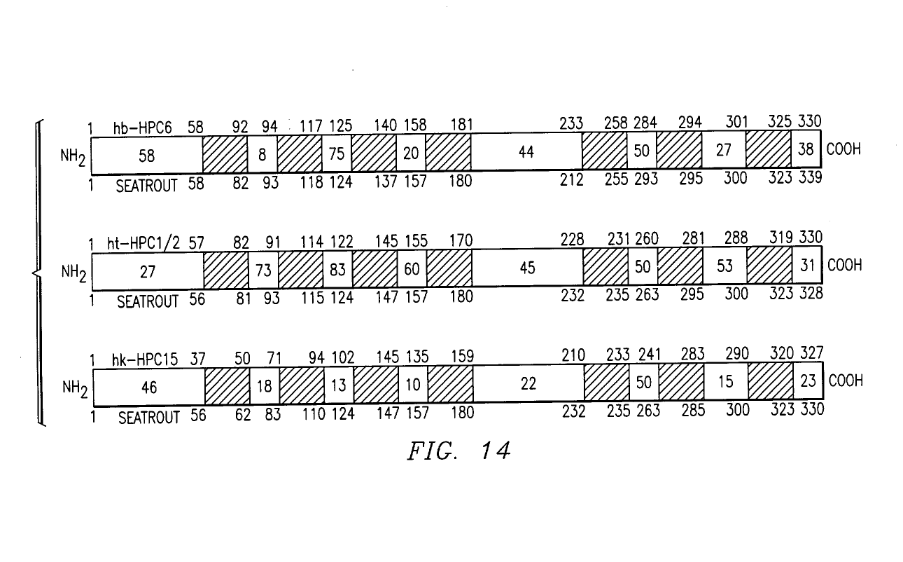

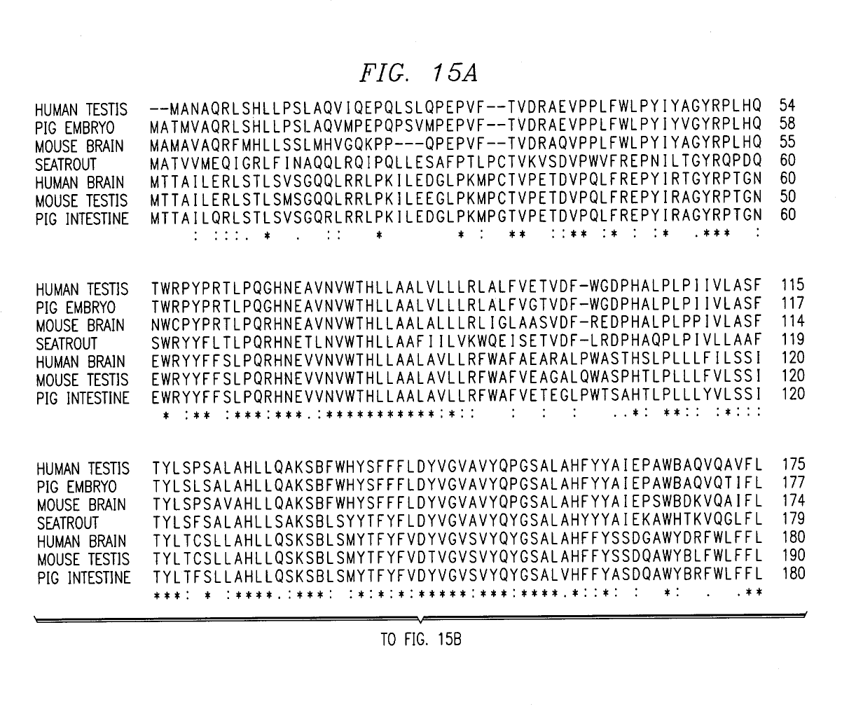

The presence of the receptor was examined in the ovary, testis, brain, pituitary, liver, heart, kidney, spleen, gills, intestine, and scales in seatrout by Northern blot analyses. Strong signals with a size of 1.4 kb were obtained in the ovary and brain (FIG. 13), whereas the testis and pituitary showed weaker signals (data not shown). No detectable signals were obtained in other tissues (data not shown).

A model for the insertion of seatrout membrane progestin receptor (mPR) in the plasma membrane based on hydrophobicity and charges of the amino acids analyzed by SOSUI (Hirokawa et al., 1998; Mitaku and Hirokawa, 1999) is shown in FIG. 7. Each circle represents 1 amino acid residue. Filled circles indicate conserved identical residues between the fish membrane progestin receptor (mPR) and a novel human kidney protein (hk, AK000197). A potential site of N-linked glycosylation is shown.

To obtain similar mammalian membrane steroid receptor sequences, the deduced amino acid sequence of seatrout mPR (SEQ ID NO: 2) was used in a Blast search of human htgs database at GenBank. Two pieces of human genome sequences with high homology to seatrout mPR were identified (FIG. 14 hb-HPC6 and ht-HPCI/2).

Several cDNA clones of mammals including human, mouse and pig were identified from dbest database in GenBank in a Blast search of the human genome sequences. All these cDNA clones have only been sequenced partly at 5′ or 3′-end. One additional similar cDNA clone with full-length sequence was identified from nr database in GenBank (FIG. 14, hk-HPC 1 5). None of these genes have been characterized previously.

Membrane Steroid Receptor cDNAs in Mammals

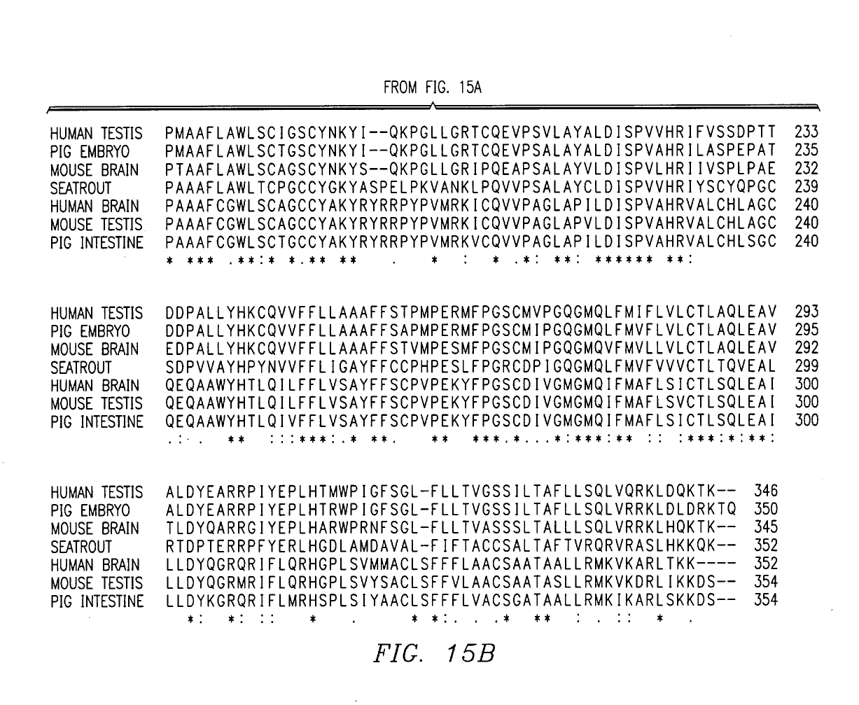

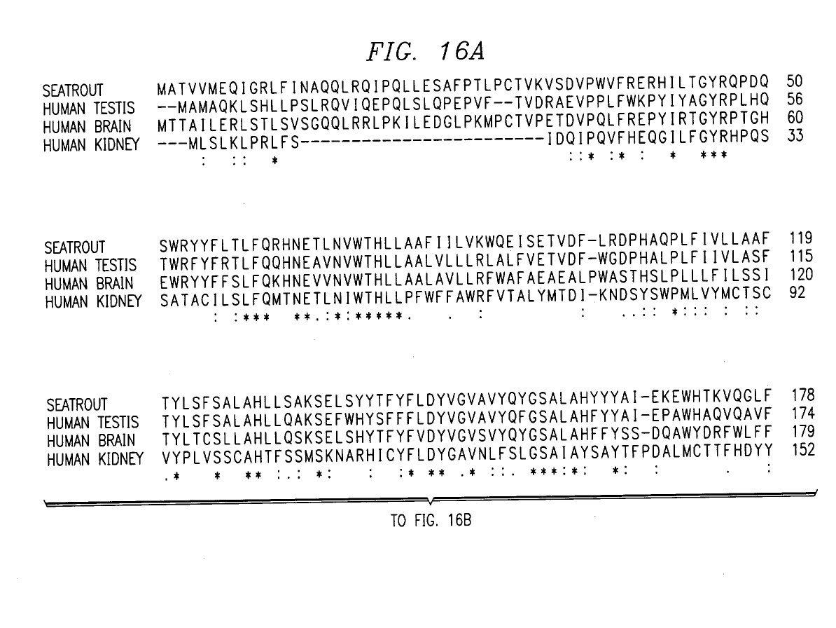

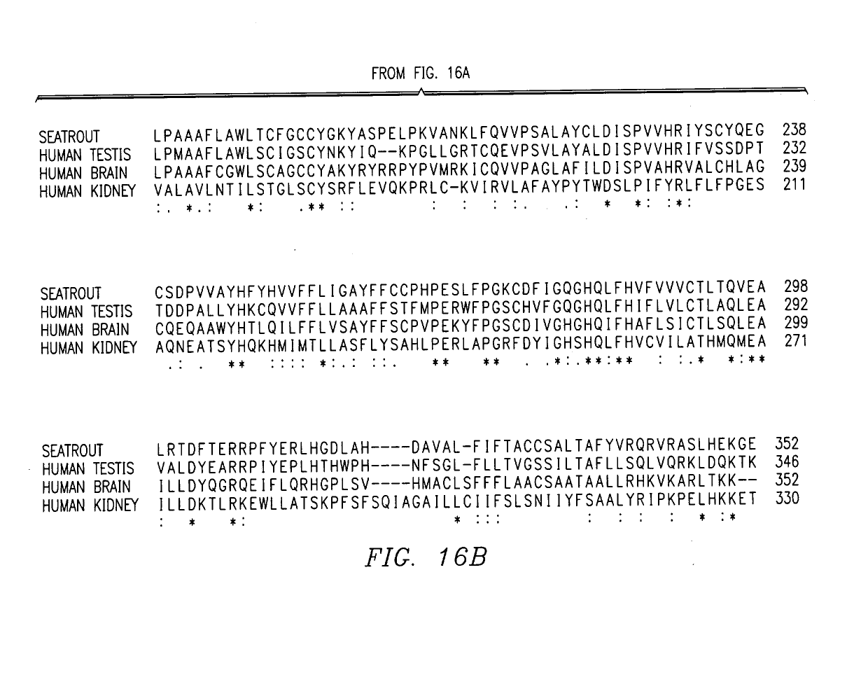

Human Testicular cDNA SEQ ID NO: 3 (ht, AF313620). Four similar EST clones from tissues of testis (dbEST Id: 1780349, 1731306), adrenal gland (dbEST Id: 990542 ) and ovary (dbEST Id:3680423) were identified in human ests database by Blast search using human genome sequence ht-HPCI/2. An IMAGE clone (dbEST 1731306, IMAGE: 1621612) from testis was selected and purchased from Research Genetics. The clone (ht) was fully sequenced, SEQ ID NO: 3, in both directions and deposited into GenBank with accession number AF313620. The full-length of the clone has 1262 nucleotides including a 22-bp poly (A) tail. The ht sequence shows an open reading frame of 1041 nucleotides starting at 97 nucleotides and ending at 1137 nucleotides position. The open reading frame encodes a polypeptide of 346 amino acid residues including a 19-aa signal peptides (SEQ ID NO: 4) (FIGS. 15, 16). The homology was 61% between ht and seatrout mPR (FIG. 28).

Human Brain cDNA SEQ ID NO: 5 (hb, AF313619). Three similar EST clones from fetal brain (dbEST Id: 448468, 448148) and neuroepithelial cells (dbEST Id: 740252) were identified in human ests database by Blast search using human genome sequence hb-HPC 6. All three clones were truncated at 5′-end. An IMAGE clone (IMAGE: 563998, dbEST Id; 740252) was purchased from Research Genetics and fully sequenced (SEQ ID NO 5).

The hb sequence has 2221 bp with 671 nucleotides encoding a 219-aa partial amino acid sequences with incomplete 5′-end (deposited into GenBank: accession number AF313619) (SEQ ID NO: 6). The hb sequence contains a large 3′-untranslated region with 4 poly(A) signals at 842, 1017, 1102 and 2182 nt position and an 18-bp poly (A) tail. The similarity was 60% between hb and fish mPR sequences (FIG. 28). The homology was 56% between hb and hi sequences (FIG. 28).

Mouse Brain cDNA SEQ ID NO: 7 (mb, AF313618).

Six similar mouse EST clones from mouse brain (dbEST Id: 3262253, 3262254, 2336487), lung (dbEST Id: 1609269), heart (dbEST Id: 779001) and embryo (dbEST Id: 735621) were identified by Blast search using ht-HPC1/2 sequence and mouse ESTs database in GenBank. A clone (dbEST Id: 3262253) was obtained from Dr. Katsuyuki Hashimoto at National Institute of Infectious Diseases, Japan. The clone (mb) was fully sequenced (SEQ ID NO: 7) in both directions and the sequence was deposited into GenBank with an accession number AF313618. The full-length of the clone has 2053 nucleotides with a poly(A) signal at 2017 nucleotides position and a 17-bp poly (A) tail. The mb sequence shows an open reading frame of 1038 nucleotides starting at 62 nucleotides and ending at position 1099 nucleotides. The open reading frame encodes a polypeptide of 345 amino acid residues (SEQ ID NO: 8) (FIG. 15). The homology of sequences was 56-82% between mb, seatrout mPR and human clones (FIG. 28).

Mouse Testis cDNA SEQ ID NO: 9 (mt, AF313617). One mouse EST clone (dbEST Id: 2469454) from mouse testis was found by Blast search using hb-HPC6 sequence and mouse ests database. An IMAGE (IMAGE Id: 567489) clone was purchased from Research Genetics and fully sequenced (SEQ ID NO: 9). The full-length of the clone (mt) has 1496 bp nucleotides with a poly (A) signal at 1476 and a 16-bp poly (A) tail. The mt sequence shows an open reading frame of 1065 nucleotides starting at 349 and ending at 1411 nt positions. The open reading frame encodes a polypeptide of 354 amino acid residues (SEQ ID NO: 10) (FIG. 15). The cDNA sequence was deposited in GenBank (accession number AF 313617). The homology was 4987% between mt and other membrane steroid receptors (FIG. 28).

Pig Embryo cDNA SEQ ID NO: 11 (pe, AF313616). One pig EST clone (dbEST Id: 3819745) was obtained from pig embryo using ht-HPC1/2 sequence and non-human and non-mouse ests database in GenBank. The clone was purchased from BACPAC Resource Center at the Children's Hospital Oakland Research Institute in Oakland Calif. The cDNA sequence (SEQ ID NO: 11) was deposited into GenBank with accession number AF313616. The full-length of the clone (pe) has 2254 hp nucleotides with a poly (A) signal at 2204 nt position and a 34-bp poly (A) tail. The pe sequence shows an open reading frame of 1053 nucleotides starting at 156 and ending at 1208 nt positions. The open reading frame encodes a polypeptide of 350 amino acid residues (SEQ ID NO: 12) (FIG. 15). The homology was 45-83% between pi and other membrane steroid receptors (FIG. 28).

Pig Intestine cDNA SEQ ID NO: 13 (pi, AF313615). One pig EST clone (dbEST Id: 1616528) obtained from pig intestine showed high homology to HPC6 sequence using Blast search of non-human and non-mouse ests database in GenBank. The clone was obtained from Dr. Merete Fredholm at The Royal Veterinary & Agricultural University Demmark. The cDNA sequence was fully sequenced (SEQ ID NO: 13) and deposited into GenBank with accession number AF313615. The full-length of the clone (pi) has 2722 bp nucleotides with a 42-bp poly (A) tail. The pi sequence shows an open reading frame of 1064 nucleotides starting at 73 and ending at 1137 nt positions. The open reading frame encodes a polypeptide of 354 amino acid residues (SEQ ID NO: 14) (FIG. 15). The homology was 49-84% between pi and other membrane steroid receptors (FIG. 28).

Human Kidney cDNA SEQ ID NO: 15 (hk, AK000197). A full-length human cDNA clone was also identified in nr database of GenBank. The clone was obtained from Dr. Sugano at Tokyo University. The homology of the hk clone was 45-49% compared to the sequence of fish mPR and pig clones (pe and pi, FIGS. 16, 17 and 28). However, homology of the hk clone was 8-19% when compared to cDNA sequences of brain and testicular clones in human and mouse. The hk clone encodes a deduced amino acid sequence (SEQ ID NO: 16), which is shown in FIG. 16 in alignment with the deduced amino acid sequences of human testicular and brain steroid membrane receptors and seatrout mSR.

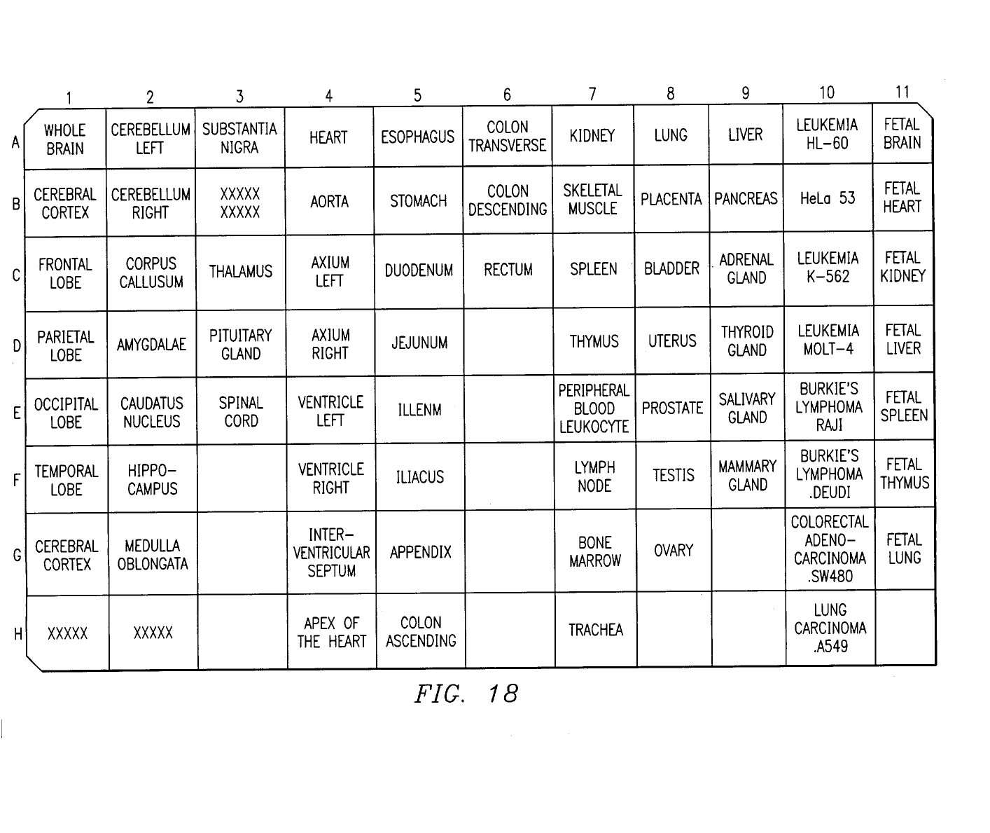

Phylogenetic analysis of the membrane steroid receptor cDNA sequences suggests that the family of genes belongs to three separate subfamilies (FIG. 17).

Localization of Membrane Steroid Receptors in Human Tissues

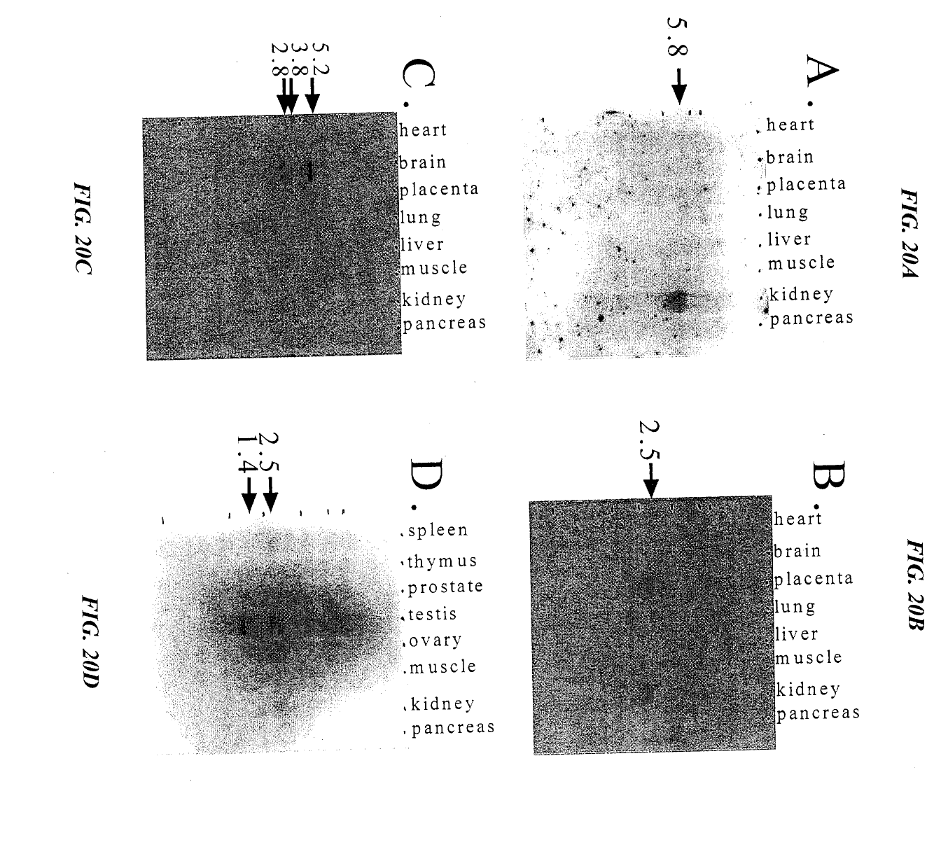

Transcripts of Human Brain Membrane Steroid Receptor (hb). Positive signals were obtained in brain, placenta and kidney in human multiple tissue arrays and multiple tissue Northern Blots using hb as a probe (FIGS. 18, 19, 20). Three hb transcripts were obtained in brain, placenta and kidney with a major signal at 5.2 kb and two minor signals at 3.2 and 2.8 kb (FIG. 20C). The transcripts were expressed strongly in brain and weakly in kidney and placenta (FIG. 20C).

Transcripts of Human Testicular Membrane Steroid Receptor (ht). Positive signals for ht were obtained in testis, ovary, placenta and kidney in human multiple tissue array and multiple tissue Northern Bolts. Two ht transcripts were obtained in testis with a major signal at 1.4 kb and a minor signal at 2,5 kb (FIG. 20D). Only the 2.5 kb transcript was obtained in ovary, placenta and kidney (FIGS. 20D, C). Transcripts were expressed strongly in testis, placenta and kidney and weakly in ovary.

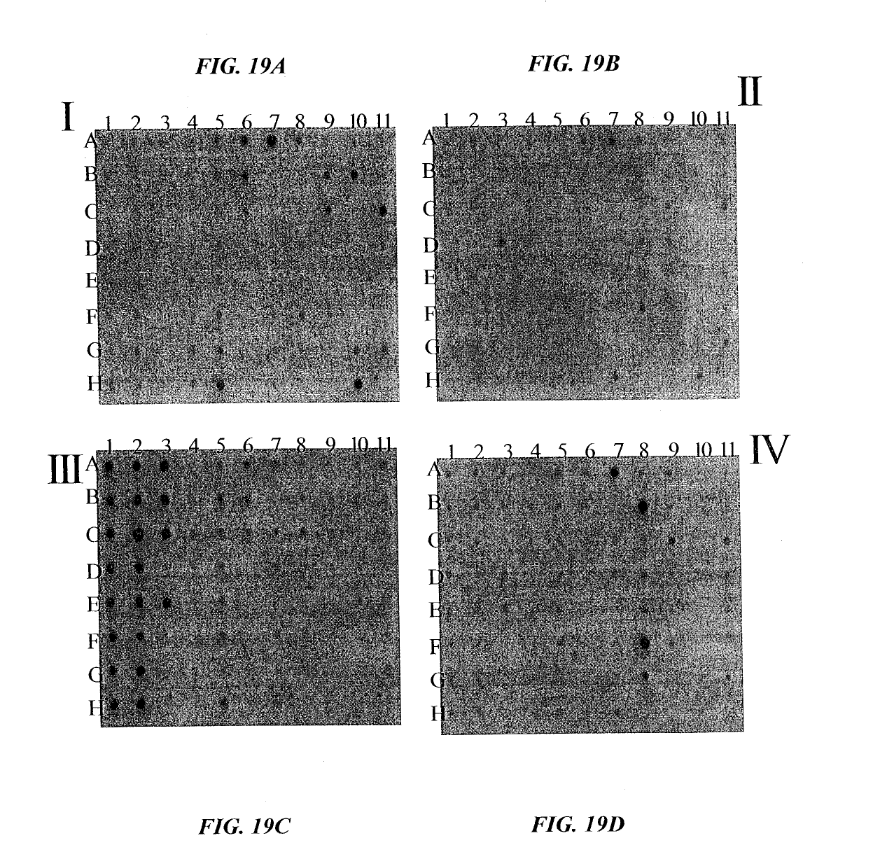

Transcripts of Human Kidney Membrane Steroid Receptors (hk). Positive signals for hk were obtained in kidney, colon, adrenal, fetal kidney, pituitary, Hela S3 and lung carcinoma A549 cells (FIG. 19, II). One transcript with 5.8 kb was obtained in the kidney (FIG. 20A).

Receptor Binding of Human Membrane Steroid Receptors. Mouse testicular and human testicular and kidney mSR were expressed in E. coli and binding of the recombinant proteins to steroids was studied. The membrane steroid receptor from human brain was not expressed since the clone only contains part of the coding region.

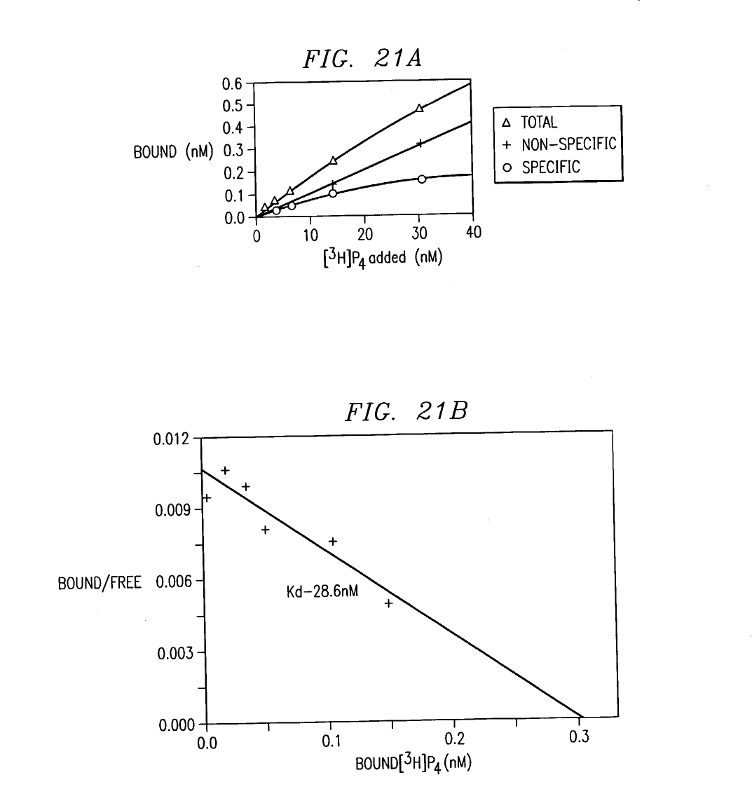

Binding Characteristics of Mouse Testicular Membrane Steroid Receptor. Saturation kinetics of ³H-progesterone binding to the recombinant mouse testicular mSR was conducted with 7 progesterone concentrations (range 0.5-40 nM) in the absence (total binding) or presence of excess cold steroid (nonspecific binding, FIG. 21). Scatchard analyses confirmed the presence of a single class of high affinity (Kd=29 nM), saturable binding sites (Bₘₐₓ=0.304 nM). The E. coli control proteins showed no specific binding to ³H-progesterone.

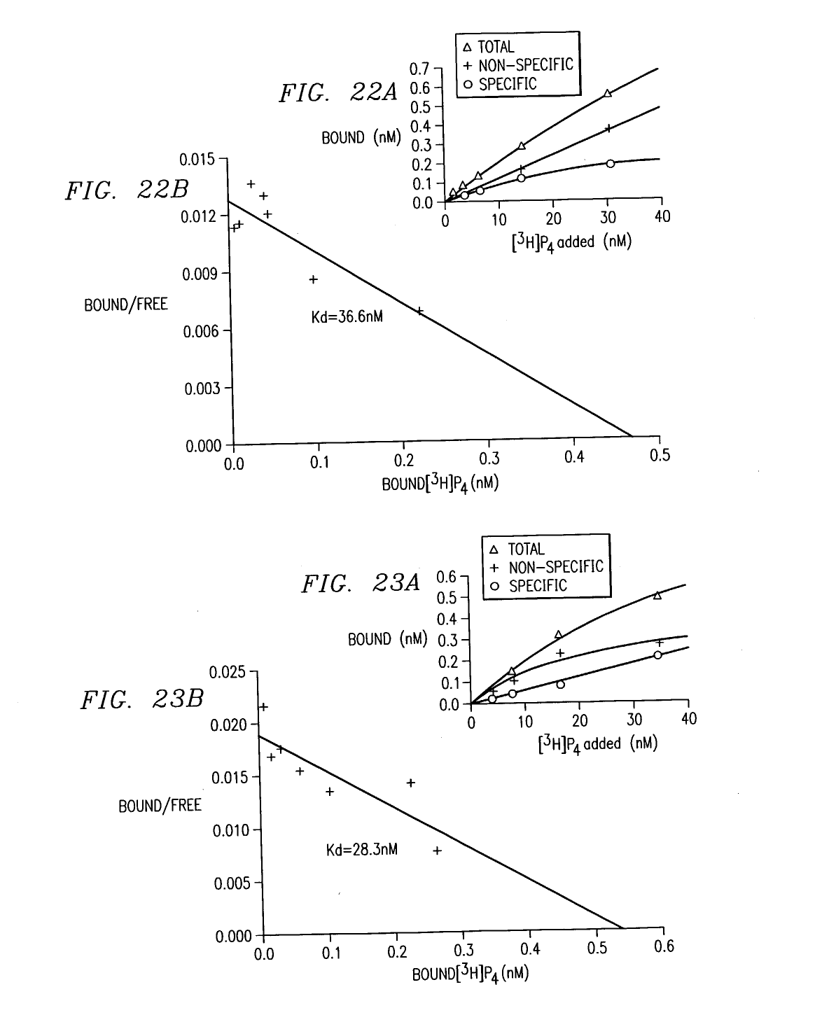

Binding Characteristics of Human Testicular Membrane Steroid Receptor. Saturation kinetics of ³H-progesterone binding to the recombinant human testicular mSR was conducted with 7 progesterone concentrations (range 0.5-40 nM) in the absence (total binding) or presence of 1 μM excess cold steroid (nonspecific binding, FIG. 22). Scatchard analyses confirmed the presence of a single class of high affinity (Kd=37 nM), saturable binding sites (Bₘₐₓ=0.47 nM). The E. coli control proteins showed no specific binding to ³H-progesterone.

Binding Characters of Human Kidney Membrane Steroid Receptor. Saturation kinetics of ³H-progesterone binding to the recombinant human kidney mSR was conducted with 7 progesterone concentrations (range 0.5-40 nM) in the absence (total binding) or presence of 1 μM excess cold steroid (nonspecific binding, FIG. 23). Scatchard analyses confirmed the presence of a single class of high affinity (Kd=28 nM), saturable binding sites (Bₘₐₓ=0.54 nM). The E. coli control proteins showed no specific binding to ³H-progesterone.

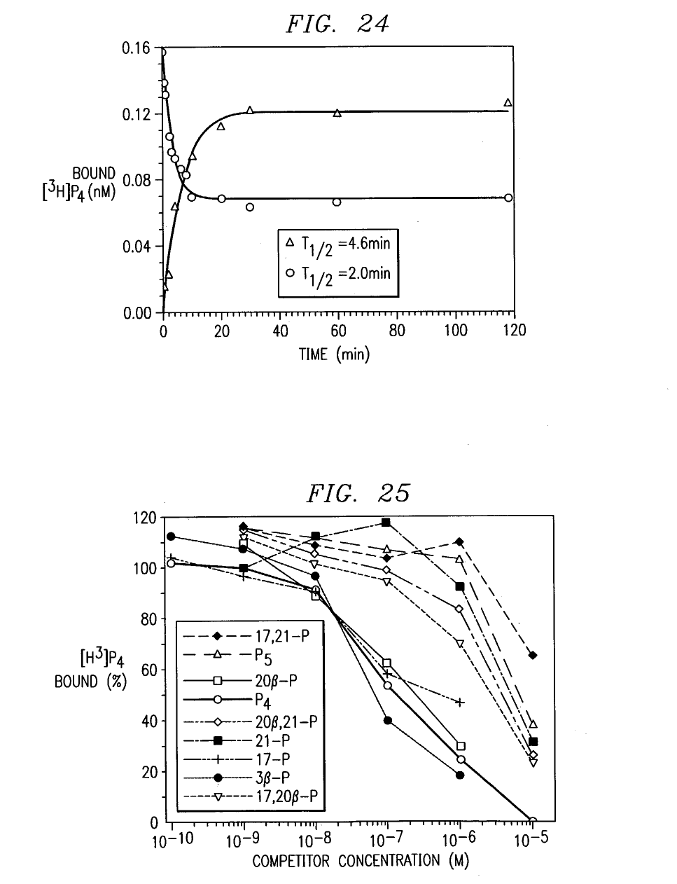

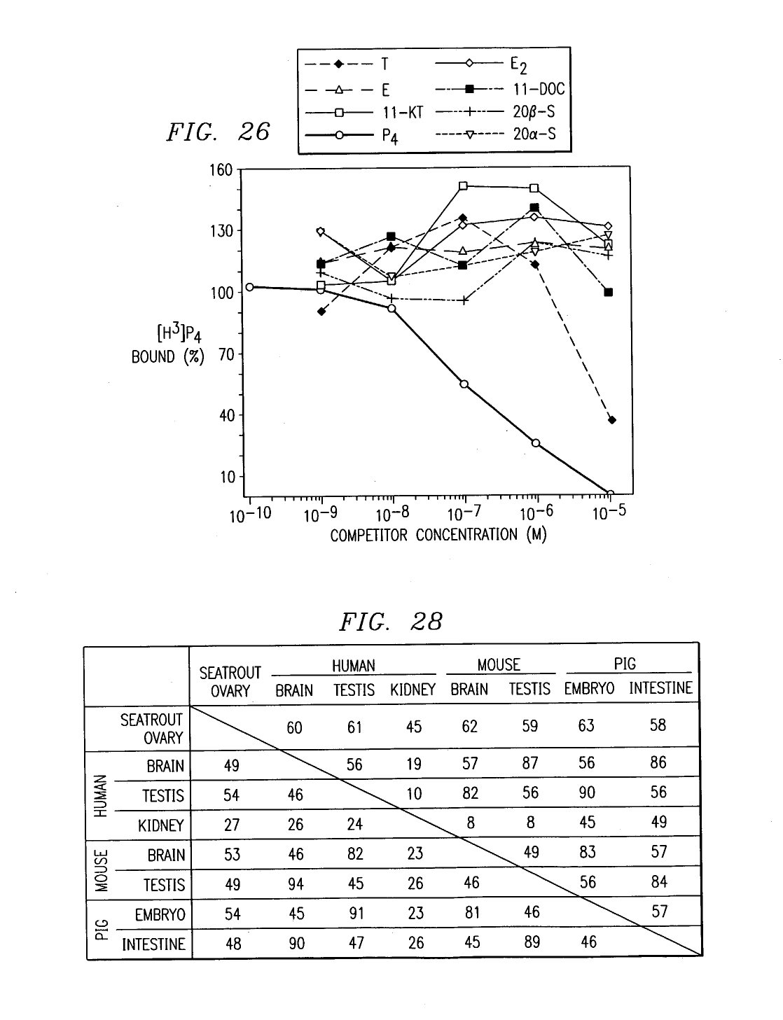

Association of the ligand for the hk steroid membrane receptor was rapid with a t_{1/2} association of 4.6 minutes. The specific binding dissociated quickly with a t_{1/2} dissociation of 2 minutes (FIG. 24).

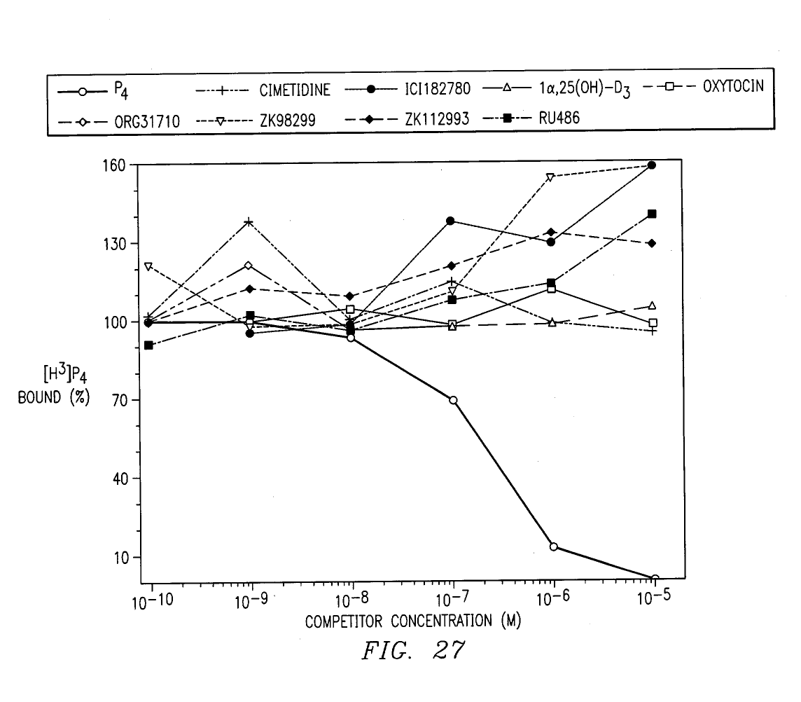

Full competition curves were generated with 10 nM 3 H-progesterone (total binding) or in combination with 1 nM-10 μM of various steroids (FIG. 25, 26). 17-P, 3β-P4 and 20β-P were equipotent with P4 in displacing ³H-progesterone. This binding was very specific for 21 carbon steroids lacking a functional group at the 11 position. Vitamin D, oxytocin and anti-hormones showed no displacement of progesterone (FIG. 27).

While the invention has been described in reference to illustrative embodiments, the description is not intended to be construed in a limiting sense. Various modifications and combinations of the illustrative embodiments, as well as other embodiments of the invention, will be apparent to persons skilled in the art upon reference to the description. It is therefore intended that the appended claims encompass any such modifications or embodiments.

What is claimed is:

1. An isolated nucleic acid sequence, and complements and homologues thereof, comprising a sequence that hybridizes under stringent conditions to a hybridization probe, wherein the nucleotide sequence of the hybridization probe comprises a nucleotide sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13 and SEQ ID NO: 15.

2. The nucleic acid sequence of claim 1, wherein the nucleic acid encodes for a membrane steroid receptor protein.

3. The nucleic acid sequence of claim 2, wherein the steroid comprises progesterone.

4. The nucleic acid sequence of claim 1, wherein the nucleic acid is found in spotted seatrout.

5. The nucleic acid sequence of claim 1, wherein the nucleic acid comprises a cDNA.

6. The nucleic acid sequence of claim 1, wherein the nucleic acid comprises an mRNA.

7. An expression vector comprising the nucleic acid sequence of claim 1.

8. A host cell comprising the expression vector of claim 7.

9. A trans-species family of genes encoding for a membrane steroid receptor, wherein each gene of the family comprises or has homology to a spotted seatrout (Cynoscion nebulosus) gene that encodes for an ovarian membrane progestin (20β-S) receptor.

10. A gene of the family of genes of claim 9, wherein the gene may be isolated from a fish.

11. A gene of the family of genes of claim 10, wherein the gene is isolated from fish ovaries.

12. A gene of the family of genes of claim 9, wherein the gene may be isolated from a pig.

13. A gene of the family of genes of claim 12, wherein the gene is isolated from pig embryonic tissue.

14. A gene of the family of genes of claim 12, wherein the gene is isolated from pig intestine.

15. A gene of the family of genes of claim 9, wherein the gene may be isolated from a mouse.

16. A gene of the family of genes of claim 15, wherein the gene is isolated from mouse brain.

17. A gene of the family of genes of claim 15, wherein the gene is isolated from mouse testis.

18. A gene of the family of genes of claim 9, wherein the gene may be isolated from a human.

19. A gene of the family of genes of claim 18, wherein the gene is isolated from human kidney.

20. A gene of the family of genes of claim 18, wherein the gene is isolated from human brain.

21. A gene of the family of genes of claim 18, wherein the gene is isolated from human testes.

22. A gene of the family of genes of claim 9, wherein the gene may be isolated from a animal belonging to a phylum selected from the group consisting of fish, pig, mouse and human.

23. A gene of the family of genes of claim 9, wherein the gene comprises a nucleic acid sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13 and SEQ ID NO: 15.

24. A purified membrane steroid receptor.

25. The purified membrane steroid receptor of claim 24, wherein the receptor is purified from a vertebrate animal.

26. The purified membrane steroid receptor of claim 25, wherein the receptor is purified from a human.

27. The purified membrane steroid receptor of claim 25, wherein the receptor is purified from a fish.

28. The purified membrane steroid receptor of claim 25, wherein the receptor is purified from a pig.

29. The purified membrane steroid receptor of claim 24, wherein the receptor is purified from a mouse.

30. The purified membrane steroid receptor of claim 24, wherein the receptor comprises an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14 and SEQ ID NO: 16.

31. A membrane steroid receptor capture protocol comprising the steps of:
forming a complex between solubilized membrane proteins and an antibody and an antigen that specifically binds to the antibody;
exposing a steroid to the complex, whereby the steroid specifically binds to the complex; and
identifying a receptor to which the steroid is specifically bound.

32. The protocol of claim 31, wherein the membrane steroid is progestin.

33. The protocol of claim 31, wherein the membrane steroid is radiolabeled.

34. The protocol of claim 31, wherein the antibody is goat anti-mouse IgG FC and the antigen is mouse IgG.

35. The protocol of claim 31, wherein the complex comprises progestin 20β-S, goat anti-mouse IgG FC and mouse IgG.

36. The protocol of claim 31, further comprising the step of generating antibodies to the receptor.

37. The protocol of claim 36, wherein the antibodies comprise monoclonal antibodies.

38. A purified membrane steroid receptor obtained by a method comprising the protocol of claim 31.

39. An antibody obtained by a method comprising the protocol of claim 36.
